(19) United States
(12) Patent Application Publication
(10) Pub. No.: US 2024/0390564 A1
(43) Pub. Date: Nov. 28, 2024

(54) DEVICE FOR PROVIDING DIALYSIS INFORMATION AND PROGRAM FOR PROVIDING DIALYSIS INFORMATION

(71) Applicant: NATIONAL UNIVERSITY CORPORATION SHIZUOKA UNIVERSITY, Shizuoka-shi, Shizuoka (JP)

(72) Inventors: Yoshihiko SANO, Hamamatsu-shi, Shizuoka (JP); Kentaro SATO, Hamamatsu-shi, Shizuoka (JP); Toyomu UGAWA, Bunkyo-ku, Tokyo (JP); Narutoshi KABASHIMA, Onga-gun, Fukuoka (JP)

(21) Appl. No.: 18/696,339

(22) PCT Filed: Sep. 29, 2021
(86) PCT No.: PCT/JP2021/035856

§ 371 Date: Mar. 27, 2024

(51) Int. Cl. A61M 1/16

(52) U.S. Cl. CPC A61M 1/1613; A61M 2230/20

(57) ABSTRACT

A device for providing dialysis information receives a target dialysis conditional value indicating the target for the dialysis among the plurality of dialysis conditions, a plurality of known dialysis conditional values excluding the search dialysis conditional value and the target dialysis conditional value from the plurality of dialysis conditional values, and one or a plurality of patient-specific values set for each patient. The device for providing dialysis information further obtains the search dialysis conditional value satisfying the target dialysis conditional value by using a predicted toxin concentration obtained by substituting a patient test value, the known dialysis condition and the patient-specific value into an extracellular fluid toxin concentration function indicating a temporal change in concentration of a toxin to be removed contained in extracellular fluid of the patient.

### TECHNICAL FIELD

The present invention relates to a device for providing dialysis information and a program for providing dialysis information.

### BACKGROUND ART

Patent Literature 1 discloses a technique for assisting in performing dialysis. The dialysis support device of Patent Literature 1 automatically creates an electronic medical record based on instructions on dialysis treatment input by a doctor. According to the automatic medical record, even in an emergency, doctor's instructions can be transmitted to a site where dialysis is performed in real time. Patent Literature 2 discloses a technique for assisting in blood purification treatment. The blood purification therapy support system of Patent Literature 2 can accurately determine whether a treatment condition needs to be changed.

Non-Patent Literatures 1, 2, and 3 each disclose a theory on dialysis. Non-Patent Literature 1 discloses an idea of modeling a human body as two components. Non-Patent Literatures 2 and 3 each disclose an idea of modeling a human body as one component.

### CITATION LIST

### Patent Literature

Patent Literature 1: Japanese Patent No. 6900752
Patent Literature 2: Japanese Patent No. 6716428

### Non-Patent Literature

Non-Patent Literature 1: Bell, R. L. Curtis, F. K., and Babb, A. L., “Analog Simulation of the Patient-Artificial Kidney System”, Transactions-American Society for Artificial Internal Organs, 11, 183-189, 1965. (Bell, R. L. Curtis, F. K. and Babb, A. L. Analog Simulation of the Patient-Artificial Kidney System, Trans. Amer. Soc. Artif. Int. Organs. 11, 183-189 (1965).)
Non-Patent Literature 2: Shinzato, T.; Nakai, S.; Fujita, Y.; Takai, I.; Morita, E.; Nakane, K.; Maeda, K. “Determination of Kt/V and protein catabolic rate using pre- and post-dialysis blood urea nitrogen concentrations) Nephron 1994 (Shinzato, T.; Nakai, S.; Fujita, Y.; Takai, I.; Morita, E.; Nakane, K.; Maeda, K. Determination of Kt/V and protein catabolic rate using pre- and post-dialysis blood urea nitrogen concentrations. Nephron 1994.)
Non-Patent Literature 3: Gotch F. A.; Sargent, J. A., A mechanistic analysis of the National Cooperative Dialysis Study (NDCs). Kidney Int. 1985. (Gotch, F. A.; Sargent, J. A. A mechanistic analysis of the National Cooperative Dialysis Study (NDCs). Kidney Int. 1985.)

### SUMMARY OF INVENTION

### Technical Problem

In order to perform dialysis, it is necessary to determine numerical values indicating several dialysis conditions based on the conditions of the patient. Dialysis conditions influence the outcome of dialysis. It is therefore desirable to set appropriate dialysis conditions for each patient undergoing dialysis.

The present invention provides a device for providing dialysis information and a program for providing dialysis information that are capable of assisting in setting appropriate dialysis conditions for each patient undergoing dialysis.

### Solution to Problem

A device for providing dialysis information according to an aspect of the present invention provides a search dialysis conditional value that is a value selected from among a plurality of dialysis conditional values indicating conditions for dialysis and satisfies a target for the dialysis. The device for providing dialysis information includes at least one processor. That at least one processor receives a target dialysis conditional value indicating the target for the dialysis among the plurality of dialysis conditions, a plurality of known dialysis conditional values excluding the search dialysis conditional value and the target dialysis conditional value from the plurality of dialysis conditional values, and one or a plurality of patient-specific values set for each patient, and obtains the search dialysis conditional value satisfying the target dialysis conditional value by using a predicted toxin concentration obtained by substituting the known dialysis condition and the patient-specific value into an extracellular fluid toxin concentration function indicating a temporal change in concentration of a toxin to be removed contained in extracellular fluid of the patient.

The device for providing dialysis information can assist in setting appropriate dialysis conditions.

In the device for providing dialysis information, that at least one processor may receive the patient-specific value and the dialysis condition to calculate a concentration of the toxin to be removed by substituting the patient-specific value and the dialysis condition into the extracellular fluid toxin concentration function. That at least one processor may output a concentration of the toxin to be removed contained in the extracellular fluid at a predetermined time point after the dialysis.

In the device for providing dialysis information, that at least one processor may receive the patient-specific value and the dialysis condition to calculate a concentration of the toxin to be removed by substituting the patient-specific value and the dialysis condition into the extracellular fluid toxin concentration function. That at least one processor may output a concentration of the toxin to be removed contained in the extracellular fluid at a predetermined time point during the dialysis.

In the device for providing dialysis information, that at least one processor may determine whether the search dialysis conditional value satisfying the target dialysis conditional value has been obtained. That at least one processor may use a result of the determination to determine whether the dialysis conditional value to be corrected is present among the plurality of received dialysis conditional values.

In the device for providing dialysis information, the patient-specific value may include a recirculation rate. That at least one processor may calculate the concentration of the toxin to be removed by using also the recirculation rate, in addition to the patient-specific value and the dialysis condition, for the extracellular fluid toxin concentration function.

In the device for providing dialysis information, the search dialysis conditional value may be a value of clearance.

In the device for providing dialysis information, the search dialysis conditional value may be a value of an index (Kt/V) defined by clearance, a dialysis time, and a total volume of body fluid.

That at least one processor may output information for obtaining the patient-specific value by using the extracellular fluid toxin concentration function.

In the device for providing dialysis information, that at least one processor may receive a test toxin concentration obtained by testing a concentration of the toxin to be removed contained in the extracellular fluid of the patient in a period in which the dialysis is performed and a concentration of the toxin to be removed contained in the extracellular fluid of the patient in a period after the dialysis is completed. That at least one processor may predict a predicted toxin concentration indicating a temporal change in the concentration of the toxin to be removed contained in the extracellular fluid by using the extracellular fluid toxin concentration function.

A deice for providing dialysis information according to another aspect of the present invention obtains one or a plurality of patient-specific values set for each patient undergoing dialysis. The device for providing dialysis information includes at least one processor. That at least one processor receives a test toxin concentration obtained by testing a concentration of the toxin to be removed contained in the extracellular fluid of the patient in a period in which the dialysis is performed and a concentration of the toxin to be removed contained in the extracellular fluid of the patient in a period after the dialysis is completed. That at least one processor obtains a predicted toxin concentration indicating a temporal change in the concentration of the toxin to be removed contained in the extracellular fluid by substituting a plurality of dialysis conditional values indicating conditions for the dialysis into an extracellular fluid toxin concentration function indicating a temporal change in the concentration of the toxin to be removed contained in the extracellular fluid of the patient.

A program for providing dialysis information according to yet another aspect of the present invention provides a search dialysis conditional value that is a value selected from among a plurality of dialysis conditional values indicating conditions for dialysis and satisfies a target for the dialysis. The program for providing dialysis information causes a computer to function as a dialysis conditional value pre-processing unit configured to receive a target dialysis conditional value indicating the target for the dialysis among the plurality of dialysis conditions, a plurality of known dialysis conditional values excluding the search dialysis conditional value and the target dialysis conditional value from the plurality of dialysis conditional values, and one or a plurality of patient-specific values set for each patient. The program for providing dialysis information causes a computer to function as a search dialysis conditional value acquisition unit configured to obtain the search dialysis conditional value satisfying the target dialysis conditional value by using a predicted toxin concentration obtained by substituting the known dialysis condition and the patient-specific value into an extracellular fluid toxin concentration function indicating a temporal change in concentration of a toxin to be removed contained in extracellular fluid of the patient.

The program for providing dialysis information may cause the computer to further function as a toxin concentration calculation unit configured to receive the patient-specific value and the dialysis condition transmitted from the dialysis conditional value pre-processing unit to calculate a concentration of the toxin to be removed by substituting the patient-specific value and the dialysis condition into the extracellular fluid toxin concentration function. The toxin concentration calculation unit may output a concentration of the toxin to be removed contained in the extracellular fluid at a predetermined time point after the dialysis.

The program for providing dialysis information may cause the computer to further function as a toxin concentration calculation unit configured to receive the patient-specific value and the dialysis condition transmitted from the dialysis conditional value pre-processing unit to calculate a concentration of the toxin to be removed by substituting the patient-specific value and the dialysis condition into the extracellular fluid toxin concentration function. The toxin concentration calculation unit may output a concentration of the toxin to be removed contained in the extracellular fluid at a predetermined time point during the dialysis.

The program for providing dialysis information may cause the computer to function as a determination unit configured to determine whether the search dialysis conditional value satisfying the target dialysis conditional value has been obtained. The program for providing dialysis information may cause the computer to further function as a correction necessity determination unit configured to use a result of the determination to determine whether the dialysis conditional value to be corrected is present among the plurality of received dialysis conditional values.

In the program for providing dialysis information, the patient-specific value may include a recirculation rate. The toxin concentration calculation unit may calculate the concentration of the toxin to be removed by using also the recirculation rate, in addition to the patient-specific value and the dialysis condition, for the extracellular fluid toxin concentration function.

In the program for providing dialysis information, the search dialysis conditional value may be a value of clearance.

In the program for providing dialysis information, the search dialysis conditional value may be a value of an index (Kt/V) defined by clearance, a dialysis time, and a total volume of body fluid.

The program for providing dialysis information may cause the computer to further function as a patient-specific value proposal unit configured to output information for obtaining the patient-specific value by using the extracellular fluid toxin concentration function.

The program for providing dialysis information may cause the computer to function as a test toxin concentration processing unit configured to receive a test toxin concentration obtained by testing a concentration of the toxin to be removed contained in the extracellular fluid of the patient in a period in which the dialysis is performed and a concentration of the toxin to be removed contained in the extracellular fluid of the patient in a period after the dialysis is completed. The program for providing dialysis information may cause the computer to further function as a predicted toxin concentration processing unit configured to predict a predicted toxin concentration indicating a temporal change in the concentration of the toxin to be removed contained in the extracellular fluid by using the extracellular fluid toxin concentration function.

A program for providing dialysis information according to yet another aspect of the present invention obtains one or a plurality of patient-specific values set for each patient undergoing dialysis. A program for providing dialysis information according to yet another aspect may cause the computer to function as a test toxin concentration processing unit configured to receive a test toxin concentration obtained by testing a concentration of a toxin to be removed contained in an extracellular fluid of the patient in a period in which the dialysis is performed and a concentration of the toxin to be removed contained in the extracellular fluid of the patient in a period after the dialysis is completed. A program for providing dialysis information according to yet another aspect may cause the computer to function as a predicted toxin concentration processing unit configured to obtain a predicted toxin concentration indicating a temporal change in the concentration of the toxin to be removed contained in the extracellular fluid by substituting a plurality of dialysis conditional values indicating conditions for the dialysis into an extracellular fluid toxin concentration function indicating a temporal change in the concentration of the toxin to be removed contained in the extracellular fluid of the patient.

### Advantageous Effects of Invention

The device for providing dialysis information and the program for providing dialysis information of the present invention can assist in setting appropriate dialysis conditions.

### BRIEF DESCRIPTION OF DRAWINGS

FIG. 1 is a functional block diagram illustrating a dialysis information providing device according to an embodiment.

FIG. 2 is an example of a result output by the dialysis information providing device of FIG. 1.

FIG. 3 is a flowchart illustrating the operation of the dialysis information providing device of FIG. 1.

FIG. 4 is a diagram illustrating an example of the physical configuration of the dialysis information providing device of FIG. 1.

FIG. 5(a) is an example of first data numerical values input to the dialysis information providing device of FIG. 1. FIG. 5(b) is an example of second data numerical values input to the dialysis information providing device of FIG. 1. FIG. 5(c) is an example of third data numerical values input to the dialysis information providing device of FIG. 1.

FIG. 6 is a flowchart illustrating the operation of a patient-specific value proposal unit included in the dialysis information providing device of FIG. 1.

FIG. 7 is a functional block diagram illustrating the details of the patient-specific value proposal unit included in the dialysis information providing device of FIG. 1.

FIG. 8 is a flowchart illustrating the operation of a search dialysis conditional value proposal unit included in the dialysis information providing device of FIG. 1.

FIG. 9 is a functional block diagram illustrating the details of the search dialysis conditional value proposal unit included in the dialysis information providing device of FIG. 1.

FIG. 10 is a flowchart illustrating the operation of a search dialysis conditional value acquisition unit.

FIG. 11 is a block diagram illustrating the configuration of a dialysis information providing program.

FIGS. 12(a), 12(b), 12(c), and 12(d) are examples of screen display displayed by a patient-specific value proposal module.

FIGS. 13(a), 13(b), and 13(c) are examples of screen display displayed by a search dialysis conditional value proposal module.

FIG. 14 is a functional block diagram illustrating a dialysis information providing device according to a modification.

FIG. 15 is a block diagram illustrating the configuration of a dialysis information providing program according to a modification.

FIGS. 16(a), 16(b), and 16(c) are examples of screen display displayed by the dialysis information providing device according to the modification.

FIG. 17 is a diagram for explanation of deriving corrected clearance.

FIG. 18(a) is a graph representing a first result of comparison between a test toxin concentration and a predicted toxin concentration. FIG. 18(b) is a graph representing a second result of comparison between a test toxin concentration and a predicted toxin concentration. FIG. 18(c) is a graph representing a third result of comparison between a test toxin concentration and a predicted toxin concentration.

### DESCRIPTION OF EMBODIMENTS

Hereinafter, embodiments of the present invention will be described in detail with reference to the accompanying drawings. In the description of the drawings, the same elements are denoted by the same reference numerals, and redundant description is omitted.

Kidneys are organs that remove toxins and excess water from the blood. A condition in which the kidney function is chronically reduced is called renal failure. The number of patients with chronic renal failure is increasing year by year. Patients with chronic renal failure undergo artificial dialysis (hereinafter simply referred to as “dialysis”). Artificial dialysis is a treatment method in which a device called a dialyzer that artificially substitutes the kidney function to purify the blood. In general, artificial dialysis is sometimes performed about three times per week, and it takes about four hours for each dialysis session.

Ideally, it is desirable to constantly measure toxin concentrations in the blood. However, at present, no technique has been established for constantly measuring toxin concentrations in the blood. In addition, drawing blood a plurality of times to measure toxin concentrations is not realistic from the viewpoint of time and cost, and the viewpoint of the burden on a patient.

Therefore, dialysis conditions are determined while estimating toxin concentrations after dialysis obtained as a result of dialysis for each patient. Dialysis conditions are influenced by various factors. It is therefore difficult to determine dialysis conditions. Until now, dialysis conditions have been determined by estimating toxin concentrations after dialysis from the experience and medical viewpoint of doctors.

Examples of the dialysis conditions include clearance. Clearance refers to the amount of blood that can be purified per unit time. Clearance is also an indicator of the performance of a dialyzer that is the primary component of a dialysis machine. Clearance is set for each patient depending on the patient's condition and characteristics. That is, the dialyzer to be used for dialysis can be selected by determining clearance.

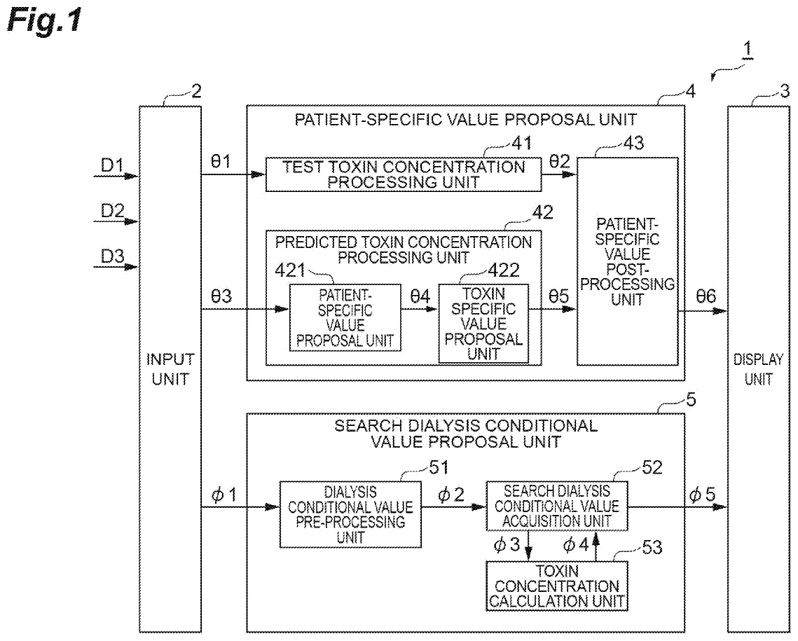

A dialysis information providing device 1 illustrated in FIG. 1 assists in determining a numerical value of dialysis conditions represented by clearance. The dialysis information providing device 1 receives an input of a target numerical value for dialysis to propose a numerical value of dialysis conditions under which the target can be achieved.

The dialysis conditions proposed by the dialysis information providing device 1 are not limited to the clearance. In addition to the clearance, the dialysis information providing device 1 can also propose any one numerical value of a dialysis time, a blood flow rate, a dialysis index (Kt/V), water removal, and a dialysate volume. The dialysis information providing device 1 can also propose two or more numerical values selected from among the dialysis time, the blood flow rate, the dialysis index (Kt/V), the water removal, and the dialysate volume. In the following description, the dialysis information providing device 1 that proposes a numerical value of the clearance will be exemplified.

In order to propose dialysis conditions, it is necessary to predict transition of a toxin concentration in a patient undergoing dialysis. Predicting the transition of the toxin concentration presents a specific problem. The problem is a rebound phenomenon. The rebound phenomenon is a phenomenon in which the toxin concentration generated after dialysis rapidly increases. The increase in toxin concentration cannot be appropriately simulated by conventional analytical models or prediction formulas based on the analytical models. Therefore, in determining dialysis conditions, the influence of the rebound phenomenon has been only considered from an empirical aspect.

The present inventors have derived Formulas (1) to (4) with which the transition of toxin concentration in a patient undergoing dialysis can be predicted accurately. Formula (1) represents a toxin concentration contained in the extracellular fluid during dialysis. Formula (2) represents a toxin concentration contained in the intracellular fluid during dialysis. Formula (3) represents a toxin concentration contained in the extracellular fluid after dialysis. That is, Formula (3) is an extracellular fluid toxin concentration function. Formula (4) represents a toxin concentration contained in the intracellular fluid after dialysis. The detailed description of Formulas (1) to (4) will be given later. Thus, the detailed description of Formulas (1) to (4) will be omitted in this paragraph.

$$\begin{matrix}[ Formula 1 ] & \\ {C}_{ex} ( t ) = {{I}_{4} [ 1 - \frac{{ω}_{f} - {ω}_{pl}}{{V}_{ex} ( 0 )} t ]}^{\frac{{λ}_{1}}{{ω}_{f} - {ω}_{pl}}} + {{I}_{5} [ 1 - \frac{{ω}_{f} - {ω}_{pl}}{{V}_{ex} ( 0 )} t ]}^{\frac{{λ}_{2}}{{ω}_{f} - {ω}_{pl}}} + \frac{S}{γ} & ( 1 )\end{matrix}$$

$$\begin{matrix}[ Formula 2 ] & \\ {C}_{in} ( t ) = \frac{Ah + CL - {ω}_{f} + {ω}_{pl} - {λ}_{1}}{Ah + {ω}_{pl}} {{I}_{4} [ 1 - \frac{{ω}_{f} - {ω}_{pl}}{{V}_{ex} ( 0 )} t ]}^{\frac{{λ}_{1}}{{ω}_{f} - {ω}_{pl}}} + \frac{Ah + CL - {ω}_{f} + {ω}_{pl} - {λ}_{2}}{Ah + {ω}_{pl}} {{I}_{5} [ 1 - \frac{{ω}_{f} - {ω}_{pl}}{{V}_{ex} ( 0 )} t ]}^{\frac{{λ}_{2}}{{ω}_{f} - {ω}_{pl}}} + \frac{Ah + CL - {ω}_{f} + {ω}_{pl}}{Ah + {ω}_{pl}} \frac{S}{γ} & ( 2 )\end{matrix}$$

$$\begin{matrix}[ Formula 3 ] & \\ {C}_{ex} = {I}_{6} exp ( - {λ}_{3} t ) + {I}_{7} + \frac{S}{{V}_{ex} ( T ) + {V}_{in} ( T )} t & ( 3 )\end{matrix}$$

$$\begin{matrix}[ Formula 4 ] & \\ {C}_{in} ( t ) = [ 1 - \frac{{V}_{ex} ( T )}{Ah} {λ}_{3} ] {I}_{6} exp ( - {λ}_{3} t ) + {I}_{7} + \frac{S}{{V}_{ex} ( T ) + {V}_{in} ( T )} [ t + \frac{{V}_{ex} ( T )}{Ah} ] & ( 4 )\end{matrix}$$

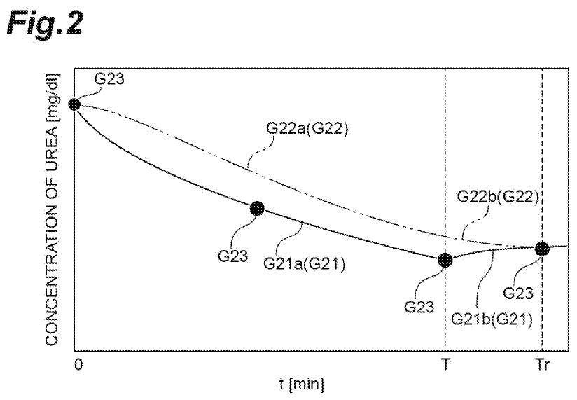

FIG. 2 illustrates temporal changes in toxin concentration obtained from Formulas (1) to (4). A graph G21 shows the toxin concentration in the extracellular fluid. A graph G22 shows the toxin concentration in the intracellular fluid. A graph G21a is obtained by Formula (1). A graph G22a is obtained by Formula (2). A graph G21b is obtained by Formula (3). A graph G22b is obtained by Formula (4). For example, as shown in the graph G21a, during dialysis (0<t<T), the toxin concentration in the extracellular fluid decreases over time. Then, as shown in the graph G21b, the toxin concentration in the extracellular fluid increases immediately after the end of dialysis (T). Then, after a predetermined time elapses, the toxin concentration in the extracellular fluid and the toxin concentration in the intracellular fluid converge to approximately the same value. It is of noted that the toxin concentration in the extracellular fluid increases immediately after dialysis. This phenomenon is referred to as the rebound phenomenon. In determining dialysis conditions, it is desirable to correctly take account of the influence of the rebound phenomenon. With Formulas (1) to (4) derived by the inventors, it is also possible to accurately predict the rebound phenomenon.

The dialysis information providing device 1 has two functions by using Formulas (1) to (4) for predicting the toxin concentration. The dialysis information providing device 1 obtains one or a plurality of patient-specific values as a first function. The patient-specific value indicates a property related to movement of a toxin to be removed in the body of a patient. For example, the patient-specific value includes a toxin production rate, a recirculation rate, an internal mass transfer coefficient, a body water percentage, and a water content ratio. The water content ratio is the ratio between the extracellular fluid and the intracellular fluid.

The dialysis information providing device 1 proposes one or a plurality of dialysis conditions as a second function. When proposing dialysis conditions, the dialysis information providing device 1 uses the patient-specific value obtained by the first function. The use of Formulas (1) to (4) and the patient-specific value enables accurate prediction of the transition of toxin concentration in a patient undergoing dialysis.

The dialysis information providing device 1 having such functions is a system capable of providing useful information to a doctor who examines dialysis conditions. The toxin concentration targeted by the dialysis information providing device 1 is, for example, urea nitrogen. However, the toxin concentration targeted by the dialysis information providing device 1 is not limited to urea nitrogen. The toxin concentration to be targeted can be an arbitrary component by appropriately setting the patient-specific value to be described later.

That is, the dialysis information providing device 1 employs Formulas (1) to (4) for predicting toxin concentrations derived from a model based on a mass transfer theory. Formulas (1) to (4) can appropriately simulate the rebound phenomenon. Therefore, the transition of concentration of urea nitrogen can be accurately predicted.

The prediction by the dialysis information providing device 1 is not limited to the transition of urea nitrogen. The dialysis information providing device 1 can accurately predict the transition of concentration of a uremic toxic substance. For example, the dialysis information providing device 1 can accurately predict the transition of concentration of creatinine, which is a uremic toxic substance. Further, the dialysis information providing device 1 can also accurately predict the transition of concentration of uric acid, which is a uremic toxic substance.

Predicting a toxin concentration requires a numerical value unique to a patient. The numerical value unique to a patient is referred to as a patient-specific value. The dialysis information providing device 1 can also assist in performing work of determining a patient-specific value by comparing transition of toxin concentration obtained by a test with predicted transition of toxin concentration.

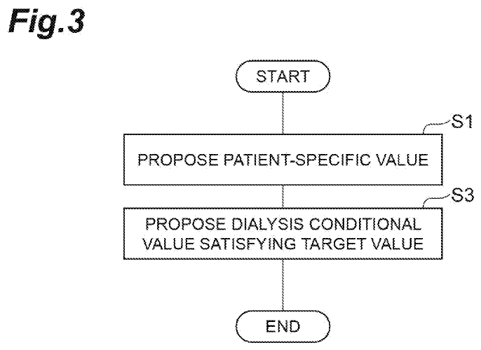

The dialysis information providing device 1 performs the operation illustrated in the flowchart of FIG. 3. In other words, a dialysis information providing program includes a description indicating the operation of FIG. 3. First, the dialysis information providing device 1 obtains a patient-specific value of a patient undergoing dialysis (Step S1). Next, the dialysis information providing device 1 proposes a dialysis condition using a plurality of numerical values including the patient-specific value (Step S3).

In the present specification, first, the details of the dialysis information providing device 1 will be described. The content of the study conducted by the inventors who derived Formulas (1) to (4) will be described after the description of the dialysis information providing device 1.

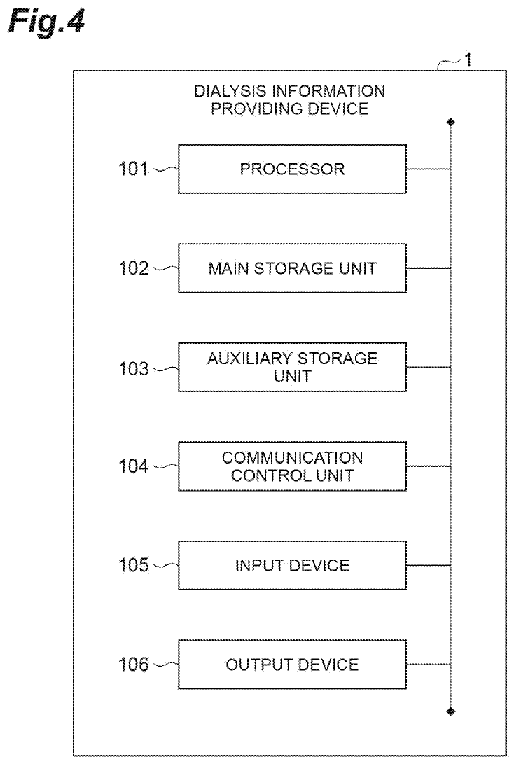

The dialysis information providing device 1 is implemented by a computer having physical constituent elements illustrated in FIG. 4. The dialysis information providing device 1 may be implemented by a single computer. The dialysis information providing device 1 may be implemented by a plurality of computers. Further, it is only required that the computer implementing the dialysis information providing device 1 has a function of inputting information, a computing function, and a function of displaying information. Examples of the computer that implements the dialysis information providing device 1 include a desktop computer, a laptop computer, and a portable computer such as a smartphone or a tablet terminal.

The computer includes a processor 101, a main storage unit 102, an auxiliary storage unit 103, a communication control unit 104, an input device 105, and an output device 106. The dialysis information providing device 1 is implemented by one or a plurality of computers including these pieces of hardware and software such as a program.

In a case where the dialysis information providing device 1 is implemented by a plurality of computers, the computers may be locally connected or may be connected via a communication network such as the Internet or an intranet. With this connection, a logically single dialysis information providing device 1 is constructed.

The processor 101 executes an operating system, an application program, and the like. The main storage unit 102 includes a read only memory (ROM) and a random access memory (RAM). The auxiliary storage unit 103 is a storage medium including a hard disk and a flash memory. The auxiliary storage unit 103 generally stores a larger amount of data than the main storage unit 102. The communication control unit 104 includes a network card or a wireless communication module. The auxiliary storage unit 103 generally stores a larger amount of data than the main storage unit 102. The input device 105 includes a keyboard, a mouse, a touch panel, and a microphone for audio input. The output device 106 includes a display and a printer.

The auxiliary storage unit 103 stores, in advance, a program and data necessary for processing. The program causes the computer to execute each functional element of the dialysis information providing device 1. By means of the program, for example, processing for providing information to assist in determining dialysis conditions is carried out in the computer. For example, the program is read by the processor 101 or the main storage unit 102 to operate at least one of the processor 101, the main storage unit 102, the auxiliary storage unit 103, the communication control unit 104, the input device 105, and the output device 106. For example, the program reads and writes data from/to the main storage unit 102 and the auxiliary storage unit 103.

The program may be provided after being recorded in a tangible storage medium such as a CD-ROM, a DVD-ROM, or a semiconductor memory. The program may be provided as a data signal via the communication network.

As illustrated in FIG. 1, the dialysis information providing device 1 includes, as functional constituent elements, an input unit 2, a display unit 3, a patient-specific value proposal unit 4, and a search dialysis conditional value proposal unit 5.

### <Input Unit>

The input unit 2 is implemented by the input device 105 illustrated in FIG. 4. The input unit 2 receives data D1, D2, and D3. The input unit 2 passes data θ1 and data θ3 to the patient-specific value proposal unit 4. The input unit 2 passes data φ1 to the search dialysis conditional value proposal unit 5.

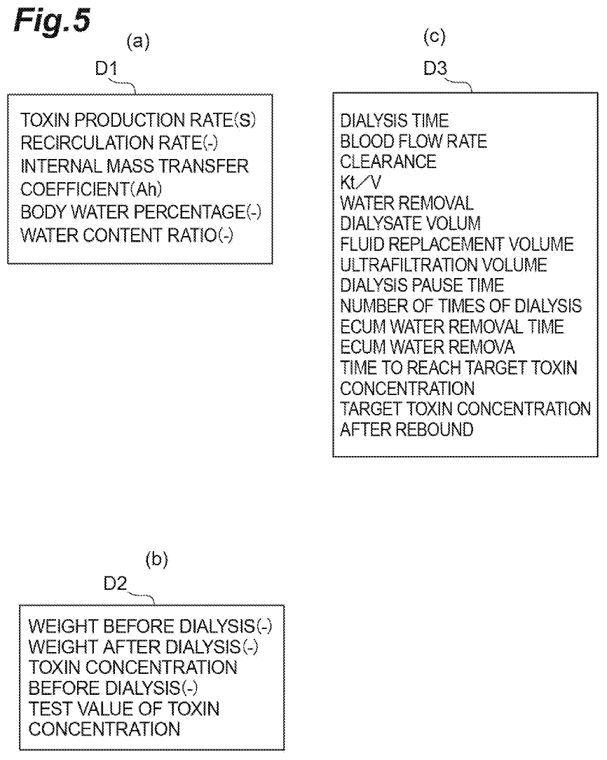

A plurality of numerical values received by the input unit 2 can be classified into several types. In the following description, information including a plurality of numerical values is referred to as “data”. FIGS. 5(a), 5(b), and 5(c) are examples of data. The content of the data handled by the dialysis information providing device 1 is not limited to the content illustrated in FIGS. 5(a), 5(b), and 5(c). The dialysis information providing device 1 is allowed to selectively handle necessary numerical values from among a plurality of numerical values illustrated in FIGS. 5(a), 5(b), and 5(c). Further, the dialysis information providing device 1 is also allowed to handle numerical values not illustrated in FIGS. 5(a), 5(b), and 5(c).

First data D1 illustrated in FIG. 5(a) includes a plurality of patient-specific values. In the present specification, as the plurality of patient-specific values, a toxin production rate, a recirculation rate, an internal mass transfer coefficient, a body water percentage, and a water content ratio are exemplified.

Here, the recirculation means the following: In a case where a position at which blood is drawn through the blood vessel is close to a position at which purified blood is returned to the blood vessel, the purified blood returned to the blood vessel is collected again. The recirculation includes the possibility of overestimating an amount of toxins removed. As described above, the dialysis information providing device 1 can handle the recirculation rate. Therefore, the possibility of overestimating the amount of toxins removed can also be eliminated. Handling the recirculation also makes it possible to confirm whether a state in which an instrument for drawing the blood and an instrument for returning the blood are attached to a patient is appropriate.

The dialysis information providing device 1 according to the present embodiment can perform computation in which the influence of recirculation is not taken into account. In the case of the computation in which the influence of recirculation is not taken into account, an input clearance value is used as it is for the computation.

Further, the dialysis information providing device 1 according to the present embodiment can perform computation in which the influence of recirculation is taken into account. In the case of the computation in which the influence of recirculation is taken into account, the value of a corrected clearance obtained by correcting the input clearance value using the value of the recirculation is used for the computation. The details of the corrected clearance will be described later.

The recirculation rate may be incorporated into the computation by correcting the clearance as described above. Alternatively, the recirculation rate may be incorporated into the computation by other methods. For example, Formulas (1) to (4) may include a term indicating the recirculation rate.

Second data D2 illustrated in FIG. 5(b) includes a plurality of test values. In the present specification, as the plurality of patient test values, a weight before dialysis, a weight after dialysis, and a toxin concentration before dialysis are exemplified. Further, the second data D2 may include a test toxin concentration. The test toxin concentration indicates a temporal change in a toxin concentration in the extracellular fluid of the patient. That is, the test toxin concentration is associated with time and toxin concentration. Dialysis (first dialysis) for obtaining the test toxin concentration is different from dialysis (second dialysis) using dialysis conditions determined by the dialysis information providing device 1. Basically, the dialysis (first dialysis) for obtaining the test toxin concentration is performed prior to the dialysis (second dialysis) using dialysis conditions determined by the dialysis information providing device 1.

Third data D3 illustrated in FIG. 5(c) includes a plurality of dialysis conditional values. In the present specification, as the plurality of dialysis conditional values, a dialysis time, a blood flow rate, clearance, a dialysis index (Kt/V), water removal, a dialysate volume, a fluid replacement volume, an ultrafiltration volume, a dialysis pause time, and the number of times of dialysis are exemplified. The plurality of dialysis conditional values may include a water removal time related to ultrafiltration and water removal related to ultrafiltration. Further, the dialysis conditional values include a target value for dialysis (dialysis target value). The dialysis target value may include, for example, a target toxin concentration after rebound and a time taken to reach the target toxin concentration.

The post-rebound target concentration indicates a toxin concentration that is the treatment target. Regarding the post-rebound target concentration, the toxin concentration rapidly changes due to the rebound phenomenon after the end of dialysis. Accordingly, in order to prevent an error between the treatment target and the actual result, a toxin concentration after the rebound phenomenon subsides is set.

The time to reach the target concentration indicates a time during which to calculate the post-rebound target concentration. This is the time for the rebound phenomenon to subside. For example, the time to reach the target concentration is generally about sixty minutes. However, the time during which a rapid change in concentration occurs due to rebound varies among individuals. In view of this, the time to reach the target concentration may be set to a value different from sixty minutes as necessary. The time to reach the target concentration after rebound as the time taken to reach the target concentration is an example. For example, it is also possible to set a target concentration at an arbitrary time during dialysis as the time taken to reach the target concentration. Further, for example, it is also possible to set a target concentration at an arbitrary time after dialysis as the time taken to reach the target concentration.

The input unit 2 may receive the data D1, D2, and D3 with the input device 105 such as a keyboard. The input unit 2 may receive the data D1, D2, and D3 from the main storage unit 102 or the auxiliary storage unit 103 included in the dialysis information providing device 1. The input unit 2 may receive the data D1, D2, and D3 from an information storage device different from the dialysis information providing device 1 via a network or the like.

### <Patient-Specific Value Proposal Unit>

The patient-specific value proposal unit 4 is implemented by executing a dialysis information support program by the processor 101 illustrated in FIG. 4.

The patient-specific value proposal unit 4 assists in determining a patient-specific value. The patient-specific value proposal unit 4 calculates a temporal change in the toxin concentration in the extracellular fluid of the patient. The temporal change in the toxin concentration output by the patient-specific value proposal unit 4 is referred to as a “predicted toxin concentration”. In calculating the predicted toxin concentration, some patient-specific values are assumed.

In a case where the assumed patient-specific values are proper, the predicted toxin concentration matches the tendency of the test toxin concentration. In a case where the assumed patient-specific values are not proper, the predicted toxin concentration does not match the tendency of the test toxin concentration. The patient-specific values are obtained by comparing the predicted toxin concentration with the test toxin concentration. The patient-specific value proposal unit 4 provides a predicted toxin concentration for comparison.

In the present specification, it is assumed that an operator who operates the dialysis information providing device 1 compares the predicted toxin concentration with the test toxin concentration. In other words, the operator inputs the assumed patient-specific value to the dialysis information providing device 1. The dialysis information providing device 1 calculates a predicted toxin concentration using the input patient-specific value. Further, the dialysis information providing device 1 displays a graph (see FIG. 2) showing both the predicted toxin concentration and the test toxin concentration on the display unit 3. The operator looks at the graph to determine the validity of the assumed patient-specific value. In a case where the assumed patient-specific value is determined to be proper, it is used to calculate the dialysis conditions. In a case where the assumed patient-specific value is determined not to be proper, the operator inputs another patient-specific value to the dialysis information providing device 1.

The patient-specific value proposal unit 4 may compare the predicted toxin concentration with the test toxin concentration. The patient-specific value proposal unit 4 sets a patient-specific value to be obtained as a search target. Then, the patient-specific value proposal unit 4 may obtain a patient-specific value leading to a predicted toxin concentration that matches the tendency of the test toxin concentration by using a predetermined search algorithm. Searching for a patient-specific value may be performed by the patient-specific value proposal unit 4 to be described later.

Further, the patient-specific value proposal unit 4 may include a means for automatically computing a patient-specific value from an analytical solution by accepting the test toxin concentration. For example, a predetermined control theory can be applied to the means for automatically computing a patient-specific value. As an example, a control theory called Newton method may be used.

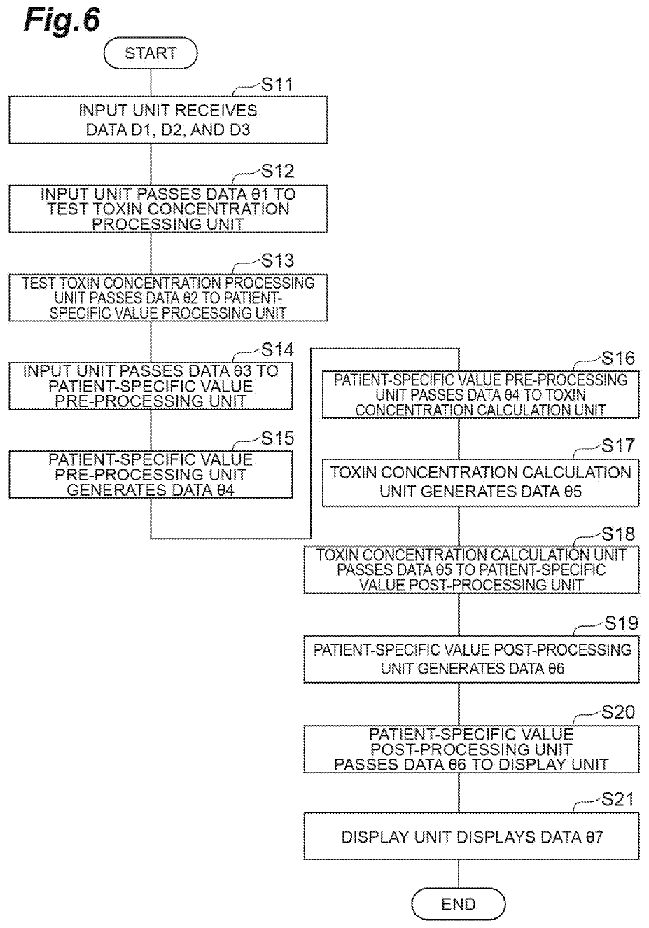

The patient-specific value proposal unit 4 includes a test toxin concentration processing unit 41, a predicted toxin concentration processing unit 42, and a patient-specific value post-processing unit 43. First, operations performed by the elements will be described with reference to the flowchart of FIG. 6. In other words, the dialysis information providing program includes a description indicating the operation of FIG. 6.

The input unit 2 receives the data D1, D2, and D3 (Step S11). Next, the input unit 2 passes the data θ1 to the test toxin concentration processing unit 41 (Step S12). Next, the test toxin concentration processing unit 41 uses the data θ1 to generate data θ2. Then, the test toxin concentration processing unit 41 passes the data θ2 to the patient-specific value post-processing unit 43 (Step S13). Next, the input unit 2 passes the data θ3 to a patient-specific value pre-processing unit 421 (Step S14). Next, the patient-specific value pre-processing unit 421 uses the data θ3 to generate data θ4 (Step S15).

Next, the patient-specific value pre-processing unit 421 passes the data θ4 to a toxin concentration calculation unit 422 (Step S16). Next, the toxin concentration calculation unit 422 uses the data θ4 to generate data θ5 (Step S17). Then, the toxin concentration calculation unit 422 passes the data θ5 to the patient-specific value post-processing unit 43 (Step S18). Next, the patient-specific value post-processing unit 43 uses the data θ5 to generate data θ6 (Step S19). Next, the patient-specific value post-processing unit 43 passes the data θ6 to the display unit 3 (Step S20). The display unit 3 then displays the data θ6 (Step S21).

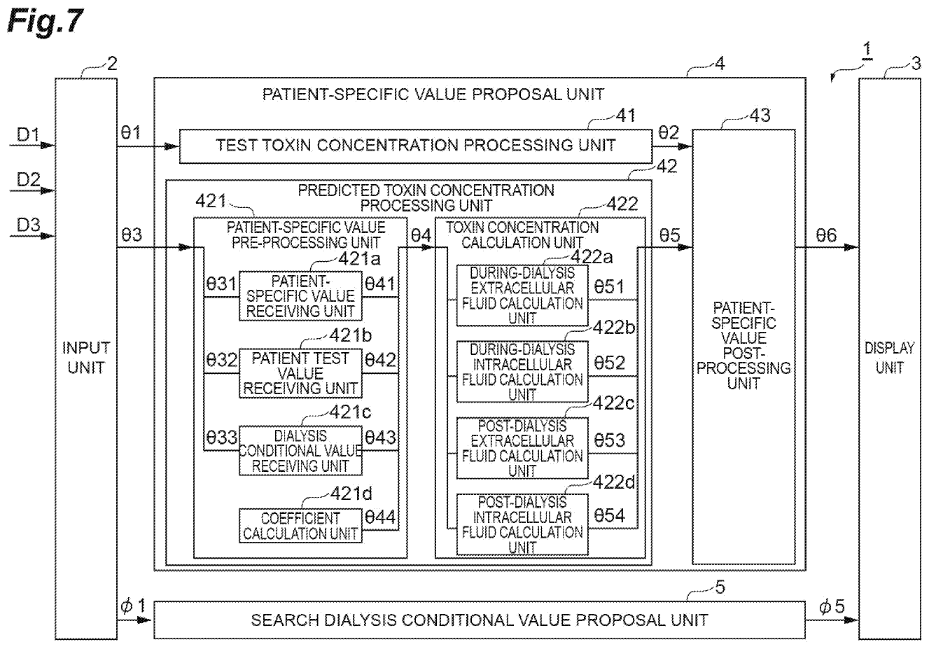

Hereinafter, the test toxin concentration processing unit 41, the predicted toxin concentration processing unit 42, and the patient-specific value post-processing unit 43 will be described in detail with reference to FIG. 7.

The test toxin concentration processing unit 41 receives the data θ1 from the input unit 2. The data θ1 is the test toxin concentration included in the data D1. The test toxin concentration processing unit 41 uses the data θ1 to obtain the data θ2. The data θ2 may be the data θ1 itself. The data θ2 may be obtained by performing predetermined computing processing on the data θ1. The test toxin concentration processing unit 41 passes the data θ2 including the test toxin concentration to the patient-specific value post-processing unit 43.

The predicted toxin concentration processing unit 42 receives the data θ3 from the input unit 2. The data θ3 includes some patient-specific values included in the data D1. The patient-specific value herein is a value obtained by using the patient-specific value proposal unit 4. The patient-specific values included in the data θ3 are assumed values. As an example, the data θ3 includes a toxin production rate in the body, a recirculation rate, and an internal mass transfer coefficient. The numerical values are values assumed by a user of the dialysis information providing device 1. Further, the data θ3 includes some patient test values included in the data D2. Specifically, the data θ3 includes a toxin concentration before dialysis, a weight before dialysis, and a weight after dialysis. Further, the data θ3 includes some dialysis conditional values included in the data D3. Specifically, the data θ3 includes clearance, a dialysis time, and a blood flow rate (dialyzer inflow rate).

The predicted toxin concentration processing unit 42 uses the data θ3 to obtain the data θ5. The operation of the predicted toxin concentration processing unit 42 that uses the data θ3 to obtain the data θ5 will be described in detail.

The predicted toxin concentration processing unit 42 includes the patient-specific value pre-processing unit 421 and the toxin concentration calculation unit 422.

The patient-specific value pre-processing unit 421 receives the data θ3. The patient-specific value pre-processing unit 421 uses the data θ3 to generate the data θ4. The data θ4 includes a numerical value required to obtain a predicted toxin concentration. The patient-specific value pre-processing unit 421 passes the data θ4 to the toxin concentration calculation unit 422. That is, the patient-specific value pre-processing unit 421 is a patient-specific value assumption unit that outputs an assumed patient-specific value that is a patient-specific value assumed.

The patient-specific value pre-processing unit 421 includes a patient-specific value receiving unit 421a, a patient test value receiving unit 421b, a dialysis conditional value receiving unit 421c, and a coefficient calculation unit 421d.

The patient-specific value receiving unit 421a receives some numerical values included in the data θ3 as data θ31. The data θ31 includes a toxin production rate in the body, a recirculation rate, and an internal mass transfer coefficient. The patient-specific value receiving unit 421a uses the data θ31 to generate data θ41. The data θ41 may be the data θ31 itself. The data θ41 may be obtained by performing predetermined computing processing on the data θ31.

The patient test value receiving unit 421b receives some numerical values included in the data θ3 as data θ32. The data θ32 includes a toxin concentration before dialysis, a weight before dialysis, and a weight after dialysis. The patient test value receiving unit 421b uses the data θ32 to generate data θ42. The data θ42 may be the data θ32 itself. The data θ42 may be obtained by performing predetermined computing processing on the data θ32.

The dialysis conditional value receiving unit 421c receives some numerical values included in the data θ3 as data θ33. The data θ33 includes clearance, a dialysis time, and a blood flow rate (dialyzer inflow rate). The dialysis conditional value receiving unit 421c uses the data θ33 to generate data θ43. The data θ43 may be the data θ33 itself. The data θ43 may be obtained by performing predetermined computing processing on the data θ33.

The coefficient calculation unit 421d uses the numerical values included in the data θ3 to calculate the values of some coefficients. The numerical value calculated by the coefficient calculation unit 421d is data θ44. The numerical value calculated by the coefficient calculation unit 421d is not included in the data θ41, θ42, and θ43. The numerical value calculated by the coefficient calculation unit 421d is generated by performing predetermined computing processing on the numerical value included in the data θ3. As an example, the data θ44 includes total body water before dialysis, an extracellular fluid volume before dialysis, an intracellular fluid volume before dialysis, an ultrafiltration volume, a ratio (εb), a blood flow rate (dialyzer outflow rate), corrected clearance, and several coefficients C₁, C₂, K₁, K₂, and λ₁.

The patient-specific value pre-processing unit 421 generates the data θ4 including the data θ41, the data θ42, the data θ43, and the data θ44. Then, the patient-specific value pre-processing unit 421 passes the data θ4 to the toxin concentration calculation unit 422.

The toxin concentration calculation unit 422 receives the data θ4 from the patient-specific value pre-processing unit 421. The toxin concentration calculation unit 422 uses the data θ4 to obtain the data θ5. The data θ5 includes a predicted value of a temporal change in the toxin concentration in the intracellular fluid and a predicted value of a temporal change in the toxin concentration in the extracellular fluid.

The toxin concentration calculation unit 422 includes a during-dialysis extracellular fluid calculation unit 422a, a during-dialysis intracellular fluid calculation unit 422b, a post-dialysis extracellular fluid calculation unit 422c, and a post-dialysis intracellular fluid calculation unit 422d.

The during-dialysis extracellular fluid calculation unit 422a receives the data θ4. The during-dialysis extracellular fluid calculation unit 422a holds Formula (1). The during-dialysis extracellular fluid calculation unit 422a obtains a function having time (t) as a variable by substituting the numerical value included in the data θ4 into Formula (1). The during-dialysis intracellular fluid calculation unit 422b substitutes the time (t) in which the range is 0 to T and the increment is Δt into the function. This yields a predicted value (data θ51) of a temporal change in the toxin concentration in the extracellular fluid during dialysis.

$$\begin{matrix}[ Formula 5 ] & \\ {C}_{ex} ( t ) = {{I}_{4} [ 1 - \frac{{ω}_{f} - {ω}_{pl}}{{V}_{ex} ( 0 )} t ]}^{\frac{{λ}_{1}}{{ω}_{f} - {ω}_{pl}}} + {{I}_{5} [ 1 - \frac{{ω}_{f} - {ω}_{pl}}{{V}_{ex} ( 0 )} t ]}^{\frac{{λ}_{2}}{{ω}_{f} - {ω}_{pl}}} + \frac{S}{γ} & ( 1 )\end{matrix}$$

The during-dialysis intracellular fluid calculation unit 422b receives the data θ4. The during-dialysis intracellular fluid calculation unit 422b holds Formula (2). The during-dialysis intracellular fluid calculation unit 422b obtains a function having time (t) as a variable by substituting the numerical value included in the data θ4 into Formula (2). The during-dialysis intracellular fluid calculation unit 422b substitutes the time (t) in which the range is 0 to T and the increment is Δt into the function. This yields a predicted value (data θ52) of a temporal change in the toxin concentration in the intracellular fluid during dialysis.

$$\begin{matrix}[ Formula 6 ] & \\ {C}_{in} ( t ) = \frac{Ah + CL - {ω}_{f} + {ω}_{pl} - {λ}_{1}}{Ah + {ω}_{pl}} {{I}_{4} [ 1 - \frac{{ω}_{f} - {ω}_{pl}}{{V}_{ex} ( 0 )} t ]}^{\frac{{λ}_{1}}{{ω}_{f} - {ω}_{pl}}} + \frac{Ah + CL - {ω}_{f} + {ω}_{pl} - {λ}_{2}}{Ah + {ω}_{pl}} {{I}_{5} [ 1 - \frac{{ω}_{f} - {ω}_{pl}}{{V}_{ex} ( 0 )} t ]}^{\frac{{λ}_{2}}{{ω}_{f} - {ω}_{pl}}} + \frac{Ah + CL - {ω}_{f} + {ω}_{pl}}{Ah + {ω}_{pl}} \frac{S}{γ} & ( 2 )\end{matrix}$$

The post-dialysis extracellular fluid calculation unit 422c receives the data θ4. The post-dialysis extracellular fluid calculation unit 422c holds Formula (3). The post-dialysis extracellular fluid calculation unit 422c obtains a function having time (t) as a variable by substituting the numerical value included in the data θ4 into Formula (3). The post-dialysis extracellular fluid calculation unit 422c substitutes time (t) in which the range is T to Tr and the increment is Δt into the function. This yields a predicted value (data θ53) of a temporal change in the toxin concentration in the extracellular fluid after dialysis.

$$\begin{matrix}[ Formula 7 ] & \\ {C}_{ex} = {I}_{6} exp ( - {λ}_{3} t ) + {I}_{7} + \frac{S}{{V}_{ex} ( T ) + {V}_{in} ( T )} t & ( 3 )\end{matrix}$$

The post-dialysis intracellular fluid calculation unit 422d receives the data θ4. The post-dialysis intracellular fluid calculation unit 422d holds Formula (4). The post-dialysis intracellular fluid calculation unit 422d obtains a function having time (t) as a variable by substituting the numerical value included in the data θ4 into Formula (4). The post-dialysis intracellular fluid calculation unit 422d substitutes time (t) in which the range is T to Tr and the increment is Δt into the function. This yields a predicted value (data θ54) of a temporal change in the toxin concentration in the intracellular fluid after dialysis.

$$\begin{matrix}[ Formula 8 ] & \\ {C}_{in} ( t ) = [ 1 - \frac{{V}_{ex} ( T )}{Ah} {λ}_{3} ] {I}_{6} exp ( - {λ}_{3} t ) + {I}_{7} + \frac{S}{{V}_{ex} ( T ) + {V}_{in} ( T )} [ t + \frac{{V}_{ex} ( T )}{Ah} ] & ( 4 )\end{matrix}$$

The toxin concentration calculation unit 422 generates the data θ5 including the data θ51 (toxin concentration in the extracellular fluid during dialysis), θ52 (toxin concentration in the intracellular fluid during dialysis), θ53 (toxin concentration in the extracellular fluid after dialysis), and θ54 (toxin concentration in the intracellular fluid after dialysis). The predicted toxin concentration processing unit 42 passes the data θ5 to the patient-specific value post-processing unit 43. It is possible that the data θ5 includes the data θ51 and the data θ53 indicating the toxin concentration in the extracellular fluid and the data θ52 and the data θ54 indicating the toxin concentration in the intracellular fluid are omitted. This is because, in determining the patient-specific value, the test toxin concentration in the extracellular fluid is compared with the predicted toxin concentration in the extracellular fluid.

The patient-specific value post-processing unit 43 obtains a graph in which the test toxin concentration and the predicted toxin concentration overlap with each other. That is, the data θ6 output by the patient-specific value post-processing unit 43 is a graph in which the test toxin concentration and the predicted toxin concentration overlap with each other.

The patient-specific value post-processing unit 43 receives the data θ2 from the test toxin concentration processing unit 41. The patient-specific value post-processing unit 43 receives the data θ5 from the predicted toxin concentration processing unit 42. The patient-specific value post-processing unit 43 generates a graph (data θ6, see FIG. 2) in which the data θ2 indicating the test toxin concentration and the data θ5 indicating the predicted toxin concentration overlap with each other. The patient-specific value post-processing unit 43 passes the data θ6 to the display unit 3.

Note that the patient-specific value post-processing unit 43 may generate information different from the graph (data θ6). For example, the patient-specific value post-processing unit 43 may generate an evaluation value that numerically indicates the degree of correspondence between the predicted toxin concentration and the test toxin concentration. The patient-specific value post-processing unit 43 may pass, as the data θ6, the evaluation value together with the graph to the display unit 3. The patient-specific value post-processing unit 43 may pass, as the data θ6, only the evaluation value to the display unit 3.

The display unit 3 is implemented by the output device 106 illustrated in FIG. 4. The display unit 3 receives the data θ6 from the patient-specific value post-processing unit 43. For example, the display unit 3 that is a display device displays the data θ6.

### <Functional Effect of Patient-Specific Value Proposal Unit>

The patient-specific value proposal unit 4 predicts a temporal change in the toxin concentration using a value including an assumed patient-specific value. Then, if the tendency of the temporal change in the predicted toxin concentration is equivalent to the tendency of the temporal change in the test toxin concentration, then it can be said that the assumed patient-specific value appropriately indicates the characteristics of the patient. That is, in the graph G21, in a case where the tendency of the temporal change in the predicted toxin concentration is different from the tendency of the temporal change in the test toxin concentration, it can be said that the assumed patient-specific value does not appropriately indicate the characteristics of the patient. Accordingly, the patient-specific value is changed by increasing or decreasing the same. Then, the graph G21 is calculated again. By repeating the tentative setting of the patient-specific value, the calculation of the graph G21 based on the patient-specific value tentatively set, and the comparison between the predicted toxin concentration and the test toxin concentration shown in the graph G21, it is possible to obtain a patient-specific value appropriately indicating the characteristics of the patient undergoing dialysis.

Specifically, the patient-specific value proposal unit 4 performs an operation of calculating the toxin concentration for each assumed patient-specific value. In the operation, the toxin concentration from the start of dialysis to the end of dialysis and the toxin concentration from immediately after the end of dialysis to a predetermined period are calculated at predetermined time intervals. The operation involves processing of calculating the toxin concentration a plurality of times.

The dialysis information providing device 1 facilitates the above-described repetitive calculations because Formulas (1) to (4) for predicting the toxin concentration are not differential equations but algebraic equations. In the case of algebraic equations, the solution to be sought can be obtained by a simple calculation method such as four arithmetic operations of numerical values. In other words, the dialysis information providing device 1 does not require enormous repetitive calculations as in the case of numerically solving a differential equation. As a result, since no enormous computational load is incurred, it can be easily mounted on any computer such as a personal computer.

### <Search Dialysis Conditional Value Proposal Unit>

The search dialysis conditional value proposal unit 5 is implemented by executing the dialysis information providing program by the processor 101 illustrated in FIG. 4.

The search dialysis conditional value proposal unit 5 assists in determining dialysis conditions. The dialysis conditions are some parameters to be determined before dialysis is performed. Examples of the main dialysis conditions include a dialysis time, a blood flow rate (flow rate into the dialyzer), and clearance. Some parameters that can be derived from the dialysis conditions may also be included as the dialysis conditions. For example, dialysis conditions that can be derived using a dialysis time, a blood flow rate (flow rate into the dialyzer), clearance, and other parameters include a dialysis index (Kt/V), water removal, and a dialysate volume.

The dialysis index (Kt/V) is determined based on the dialysis time, clearance, and body fluid volume of the patient. The water removal is determined based on a weight of the patient before dialysis and a weight of the patient after dialysis. The dialysate volume is determined by referring to a catalogue of the dialysis machine based on the blood flow rate (flow rate into the dialyzer) and the clearance. The dialysate volume is, for example, 500 milliliters per minute.

The search dialysis conditional value proposal unit 5 gives an answer to the question “Which numerical value do the specified parameters need to be set to meet the target?”. In the following description, among the plurality of dialysis conditions, those set as the question are referred to as “search dialysis conditional values” for convenience. That is, the search dialysis conditional value is a value that a user of the dialysis information providing device 1 requests the dialysis information providing device 1 to make a proposal. Among the plurality of dialysis conditions, those not set as the question are referred to as “known dialysis conditions” for convenience. The value of the known dialysis condition can be determined or has already been determined without a proposal of the dialysis information providing device 1.

In the present specification, an answer to the question “Which numerical value does the clearance need to be set to achieve a target toxin concentration in the extracellular fluid after a predetermined time has elapsed since the end of dialysis?” is given. That is, the search dialysis conditional value is clearance. The known dialysis conditions are, for example, the dialysis time and the blood flow rate (flow rate into the dialyzer). The type of the target dialysis condition, the target parameter, and the numerical value (target value) of the parameter may be freely selected from among the parameters included in Formulas (1) to (4).

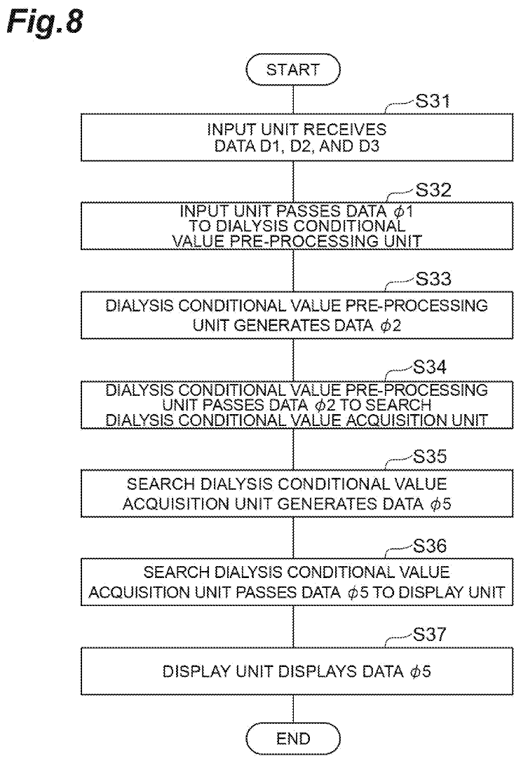

As illustrated in FIG. 1, the search dialysis conditional value proposal unit 5 includes a dialysis conditional value pre-processing unit 51, a search dialysis conditional value acquisition unit 52, and a toxin concentration calculation unit 53. First, operations performed by the elements will be described with reference to the flowchart of FIG. 8. In other words, the dialysis information providing program includes a description indicating the operation of FIG. 8.

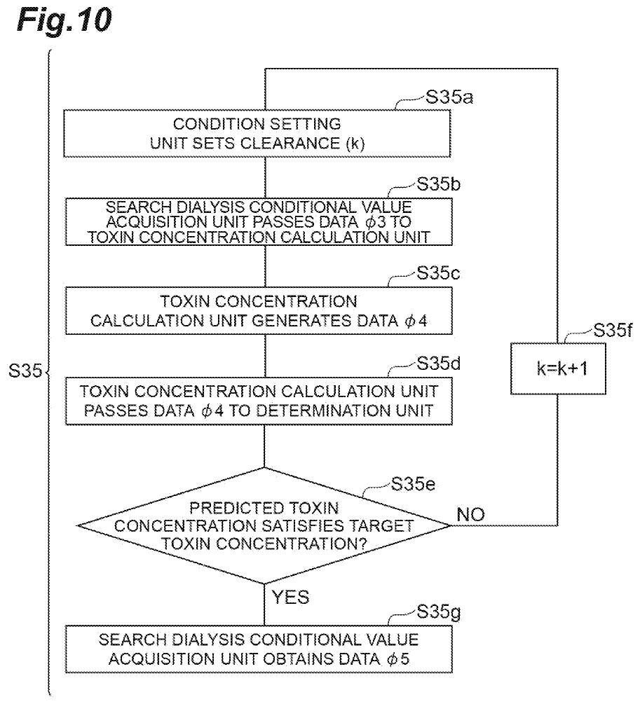

The input unit 2 receives the data D1, D2, and D3 (Step S31). Next, the input unit 2 passes the data φ1 to the dialysis conditional value pre-processing unit 51 (Step S32). Next, the dialysis conditional value pre-processing unit 51 uses the data φ1 to generate data 2 (Step S33). Next, the dialysis conditional value pre-processing unit 51 passes the data φ2 to the search dialysis conditional value acquisition unit 52 (Step S34). Next, the search dialysis conditional value acquisition unit 52 uses data φ23 and data φ4 to generate data φ5 that is a search dialysis conditional value (Step S35). The detailed processes of Step S35 are illustrated in the flowchart of FIG. 10, and will be described later. Next, the search dialysis conditional value acquisition unit 52 passes the data φ5 to the display unit 3 (Step S36). The display unit 3 then displays the data φ5 (Step S37).

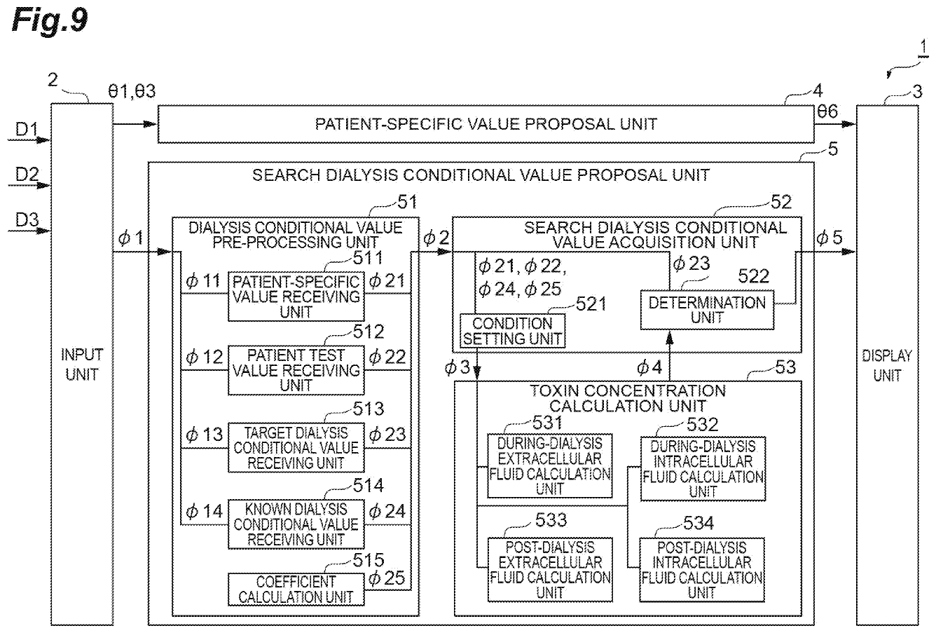

Hereinafter, the dialysis conditional value pre-processing unit 51, the search dialysis conditional value acquisition unit 52, and the toxin concentration calculation unit 53 will be described in detail with reference to FIG. 9.

The dialysis conditional value pre-processing unit 51 receives the data φ1 from the input unit 2. The dialysis conditional value pre-processing unit 51 uses the data φ1 to generate data φ2. The dialysis conditional value pre-processing unit 51 passes the data φ2 to the search dialysis conditional value acquisition unit 52.

In a case where the input unit 2 is the communication control unit 104, the dialysis conditional value pre-processing unit 51 may obtain the data φ1 via a wired or wireless network. That is, the dialysis information providing device 1 may be connected to a medical server system, a medical database system, and the like via the network. Then, the dialysis information providing device 1 may obtain the data φ1 stored in the database or system in the form of an electronic medical record.

That is, it is not always necessary that a patient-specific value used by the search dialysis conditional value proposal unit 5 is a numerical value calculated by the patient-specific value proposal unit 4. For example, the data φ1 used by the search dialysis conditional value proposal unit 5 may be selected from among a plurality of typical patient-specific values prepared in advance. The search dialysis conditional value proposal unit 5 can predict the toxin concentration more accurately when using a numerical value calculated by the patient-specific value proposal unit 4 than when using a typical patient-specific value. In other words, the search dialysis conditional value proposal unit 5 can suggest more suitable dialysis conditions when using a numerical value calculated by the patient-specific value proposal unit 4.

Further, the data φ1 used by the search dialysis conditional value proposal unit 5 may be a data set including the past dialysis data φf the patient and the patient-specific values acquired in the past. The data set includes gender, age, weight, medical history, and other relevant measurement data.

The dialysis conditional value pre-processing unit 51 includes a patient-specific value receiving unit 511, a patient test value receiving unit 512, a target dialysis conditional value receiving unit 513, and a known dialysis conditional value receiving unit 514.

The data φ1 received by the dialysis conditional value pre-processing unit 51 includes data φ11, φ12, φ13, and φ14.

The patient-specific value receiving unit 511 receives the data φ11. The data φ11 includes a plurality of patient-specific values. Specifically, the data φ11 includes a toxin production concentration, a recirculation rate, and an internal mass transfer coefficient. The patient-specific value receiving unit 511 uses the data φ11 to generate data φ21. The data φ21 may be the data φ11 itself. The data φ21 may be obtained by performing predetermined computing processing on the data φ11. The patient-specific value receiving unit 511 outputs the data φ21.

The patient test value receiving unit 512 receives the data φ12. The data φ12 includes a plurality of patient test values. Specifically, the data φ12 includes a toxin concentration before dialysis, a weight before dialysis, and a weight after dialysis. The patient test value receiving unit 512 uses the data φ12 to generate data 22. The data φ22 may be the data φ12 itself. The data φ22 may be obtained by performing predetermined computing processing on the data φ12. The patient test value receiving unit 512 outputs the data φ21.

The target dialysis conditional value receiving unit 513 receives the data φ13. The data φ13 includes a plurality of target dialysis conditional values. Specifically, the data φ13 includes a target toxin concentration after rebound and a time to reach the target toxin concentration. The target toxin concentration is the toxin concentration in the extracellular fluid. Since the target toxin concentration is the target toxin concentration after rebound, the target toxin concentration is the toxin concentration in the extracellular fluid after dialysis. The time to reach the target toxin concentration is based on the time when the dialysis is completed. The target dialysis conditional value receiving unit 513 uses the data φ13 to generate data φ23. The data φ23 may be the data φ13 itself. The data φ23 may be obtained by performing predetermined computing processing on the data φ13. The patient test value receiving unit 512 outputs the data 23.

The known dialysis conditional value receiving unit 514 receives the data φ14. The data φ14 includes a plurality of known dialysis conditional values. Specifically, the data φ14 includes a dialysis time and a blood flow rate (flow rate into the dialyzer). Among the dialysis conditions, the clearance is set as the search dialysis conditional value, and thus it is not necessary to input a numerical value. The known dialysis conditional value receiving unit 514 uses the data φ14 to generate data φ24. The data φ24 may be the data φ14 itself. The data φ24 may be obtained by performing predetermined computing processing on the data φ14. The known dialysis conditional value receiving unit 514 outputs the data φ24.

A coefficient calculation unit 515 calculates the values of some coefficients using the numerical values included in the data φ1. The numerical value calculated by the coefficient calculation unit 515 is data φ25. The numerical value calculated by the coefficient calculation unit 515 is not included in the data φ21, φ22, φ23, and φ24. The numerical value calculated by the coefficient calculation unit 515 is generated by performing predetermined computing processing on the numerical value included in the data φ1. As an example, the data φ25 includes total body water before dialysis, an extracellular fluid volume before dialysis, an intracellular fluid volume before dialysis, an ultrafiltration volume, a plasma filling amount (ωplt), a ratio (εb), a blood flow rate (dialyzer outflow rate), some coefficients K1 (after dialysis), K2 (after dialysis), λ₁ (after dialysis), a rebound calculation time, and a corrected clearance calculation result.

The dialysis conditional value pre-processing unit 51 generates the data φ2 including the data φ21, the data φ22, the data φ23, the data φ24, and the data φ25. The dialysis conditional value pre-processing unit 51 then passes the data φ2 to the search dialysis conditional value acquisition unit 52.

The search dialysis conditional value acquisition unit 52 searches for a search dialysis conditional value that satisfies the target. The search dialysis conditional value acquisition unit 52 performs the operation illustrated in the flowchart of FIG. 10. In other words, the dialysis information providing program includes a description indicating the operation of FIG. 10. First, the search dialysis conditional value acquisition unit 52 sets an assumed value of clearance that is a search dialysis conditional value (Step S35a). Next, the search dialysis conditional value acquisition unit 52 passes data including the assumed value of clearance to the toxin concentration calculation unit 53 (Step S35b). Next, the toxin concentration calculation unit 53 generates the data φ4 (Step S35c). Next, the toxin concentration calculation unit 53 passes the data φ4 to a determination unit 522 of the search dialysis conditional value acquisition unit 52 (Step S35d). Next, the determination unit 522 compares the predicted toxin concentration with the target toxin concentration (Step S35e). If it can be determined that the predicted toxin concentration satisfies the target toxin concentration (Step S35e: YES), then the assumed value is employed as the search dialysis conditional value (Step S35g). On the other hand, if it cannot be determined that the predicted toxin concentration satisfies the target toxin concentration (Step S35e: NO), then the search dialysis conditional value acquisition unit 52 changes the assumed value of the clearance (Step S35f).

The search dialysis conditional value acquisition unit 52 receives the data φ2 from the dialysis conditional value pre-processing unit 51. The search dialysis conditional value acquisition unit 52 uses the data φ2 to generate the data φ5. The data φ5 is a search dialysis conditional value that satisfies the target. The search dialysis conditional value acquisition unit 52 passes the data φ5 to the display unit 3.

The search dialysis conditional value acquisition unit 52 includes a condition setting unit 521 and the determination unit 522.

The condition setting unit 521 executes Steps S35a and S35b in the flowchart of FIG. 10. The condition setting unit 521 generates data φ3 to be delivered to the toxin concentration calculation unit 53. The condition setting unit 521 receives the data φ21, φ22, φ24, and φ25 of the data φ2. The condition setting unit 521 generates an assumed value (data φ26) of the search dialysis conditional value. The condition setting unit 521 generates the data φ3 including the data φ21, φ22, φ24, φ25, and φ26. The condition setting unit 521 passes the data φ3 to the toxin concentration calculation unit 53.

The determination unit 522 executes Steps S35e, S35f, and S35g in the flowchart of FIG. 10. The determination unit 522 receives the data φ23 of the data φ2. The data φ23 is the target toxin concentration. The determination unit 522 then receives the data φ4 from the toxin concentration calculation unit 53. The data 94 is the predicted toxin concentration in the extracellular fluid after dialysis determined based on the data 3.

The determination unit 522 compares the predicted toxin concentration that is the data φ4 with the target toxin concentration that is the data φ3. Specifically, it is determined whether the predicted toxin concentration satisfies the target toxin concentration. For example, a configuration is possible in which, in a case where the predicted toxin concentration matches the target toxin concentration, it is determined that the predicted toxin concentration satisfies the target toxin concentration, and in a case where the predicted toxin concentration does not match the target toxin concentration, it is determined that the predicted toxin concentration does not satisfy the target toxin concentration. In addition, a predetermined tolerance range is set based on the target toxin concentration. A configuration is possible in which, in a case where the tolerance range includes the predicted toxin concentration, it is determined that the target toxin concentration is satisfied, and in a case where the tolerance range does not include the predicted toxin concentration, it is determined that the target toxin concentration is not satisfied.

In a case where it is determined that the predicted toxin concentration satisfies the target toxin concentration, the determination unit 522 sets, as the search dialysis conditional value (data φ5), the assumed value from which the predicted toxin concentration has been calculated. The determination unit 522 passes the data φ5 to the display unit 3.

The toxin concentration calculation unit 53 receives the data φ3 from the search dialysis conditional value acquisition unit 52. The toxin concentration calculation unit 53 uses the data φ3 to obtain the data φ4. As described above, the data φ4 is the predicted toxin concentration in the extracellular fluid after dialysis determined based on the data φ3.

The search dialysis conditional value acquisition unit 52 may perform computation of directly deriving a solution from an analytical solution. In a case where the analytical solution is complicated, a solution can be obtained by using a control theory called Newton method. According to this configuration, the processing of obtaining the search dialysis conditional value is automatically obtained by the operation of the computer.

The toxin concentration calculation unit 53 includes a during-dialysis extracellular fluid calculation unit 531, a during-dialysis intracellular fluid calculation unit 532, a post-dialysis extracellular fluid calculation unit 533, and a post-dialysis intracellular fluid calculation unit 534. They are essentially the same as the toxin concentration calculation unit 422 of the patient-specific value proposal unit 4. Specifically, the during-dialysis extracellular fluid calculation unit 531 holds Formula (1). The during-dialysis intracellular fluid calculation unit 532 holds Formula (2). The post-dialysis extracellular fluid calculation unit 533 holds Formula (3). The post-dialysis intracellular fluid calculation unit 534 holds Formula (4). The toxin concentration calculation unit 53 substitutes the value included in the data φ3 to return the predicted toxin concentration as a return value.

In contrast, in the patient-specific value proposal unit 4, the time (t) in which the range is time (0) to time (T) and the increment is Δt is substituted into the function. In the search dialysis conditional value proposal unit 5, the time at which to obtain the predicted toxin concentration is determined. Specifically, it is the time to reach the target toxin concentration included in the data φ13. That is, the search dialysis conditional value proposal unit 5 does not need to obtain a temporal change in the toxin concentration. The toxin concentration calculation unit 53 of the search dialysis conditional value proposal unit 5 calculates only a toxin concentration at time when the target toxin concentration is reached.

The difference in the time (t) described above is based on the operation of a preceding element that generates the data φ3 to be input to the toxin concentration calculation unit 53. In other words, the toxin concentration calculation unit 53 only returns the data φ4 as the return value in response to the received data φ3. Therefore, the toxin concentration calculation unit 53 of the patient-specific value proposal unit 4 and the toxin concentration calculation unit 53 of the search dialysis conditional value proposal unit 5 have the same function. Thus, redundant descriptions will be omitted.

In the present embodiment, the configuration in which the patient-specific value proposal unit 4 includes the toxin concentration calculation unit 422 and the search dialysis conditional value proposal unit 5 includes the toxin concentration calculation unit 53 has been exemplified. As described above, since the toxin concentration calculation unit 422 and the toxin concentration calculation unit 53 are essentially the same, the dialysis information providing device 1 can employ a configuration including one shared toxin concentration calculation unit. In such a case, the shared toxin concentration calculation unit receives the data θ4 from the patient-specific value proposal unit 4 and returns the data θ5 to the patient-specific value proposal unit 4. Further, the shared toxin concentration calculation unit receives the data φ3 from the search dialysis conditional value proposal unit 5 and returns the data φ4 to the search dialysis conditional value proposal unit 5.

It is only required that the search dialysis conditional value proposal unit 5 predicts the toxin concentration after dialysis. Therefore, in the toxin concentration calculation unit 53 of the search dialysis conditional value proposal unit 5, the post-dialysis extracellular fluid calculation unit 533 receives the data φ3 and returns the data φ4 (predicted toxin concentration in the extracellular fluid after dialysis). In a case where the predicted toxin concentration in the extracellular fluid during dialysis, the predicted toxin concentration in the intracellular fluid during dialysis, and the predicted toxin concentration in the intracellular fluid after dialysis are required to calculate the predicted toxin concentration in the post-dialysis extracellular fluid calculation unit 533, the during-dialysis extracellular fluid calculation unit 531, the during-dialysis intracellular fluid calculation unit 532, and the post-dialysis intracellular fluid calculation unit 534 may be caused to perform computation.

### <Functional Effect of Search Dialysis Conditional Value Acquisition Unit>

The calculation for obtaining dialysis condition information also requires processing of calculating toxin concentrations a plurality of times, similarly to the case where the operation of calculating a patient-specific value requires processing of calculating toxin concentrations a plurality of times. The dialysis information providing device 1 of the present embodiment facilitates the above-described repetitive calculations because Formulas (1) to (4) for calculating the toxin concentration are not differential equations but algebraic equations. In the case of algebraic equations, the solution to be sought can be obtained by a simple calculation method such as four arithmetic operations of numerical values. In other words, the dialysis information providing device 1 does not require enormous repetitive calculations as in the case of numerically solving a differential equation. As a result, since no enormous computational load is incurred, it can be easily mounted on any computer such as a personal computer.

The dialysis information providing device 1 described so far has a function of determining a patient-specific value and a function of proposing dialysis conditions. According to the function of determining a patient-specific value, it is possible to obtain a patient-specific value with which to grasp the mass transfer characteristics in the body of a patient and to reproduce the temporal transition of the concentration of urea nitrogen. Further, according to the function of proposing dialysis conditions, the function of the toxin concentration calculation unit 53 can be used to present dialysis conditions necessary to achieve the target for dialysis.

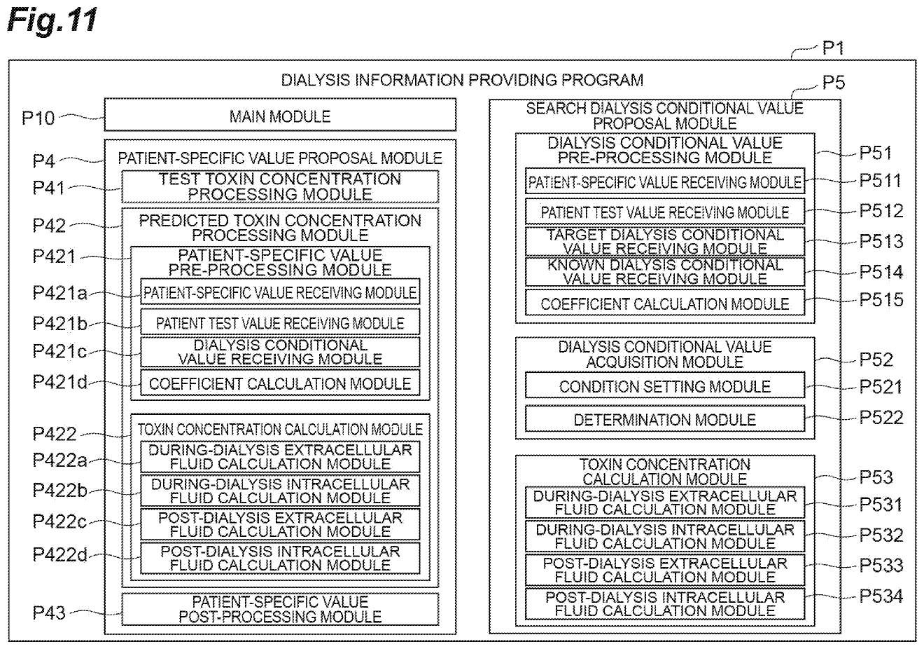

Next, an evaluation program for causing a computer to function as the dialysis information providing device 1 will be described with reference to FIG. 11.

A dialysis information providing program P1 is provided by, for example, a computer-readable recording medium such as a CD-ROM, a DVD, or a ROM, or a semiconductor memory. The dialysis information providing program P1 may be provided via the network as a computer data signal superimposed on a carrier wave.

The dialysis information providing program P1 includes a main module P10, a patient-specific value proposal module P4, and a search dialysis conditional value proposal module P5.

The main module P10 is a part that comprehensively controls the operation of the dialysis information providing device 1. The function implemented by executing the patient-specific value proposal module P4 is the same as the function of the patient-specific value proposal unit 4. The function implemented by executing the search dialysis conditional value proposal module P5 is the same as the function of the search dialysis conditional value proposal unit 5.

The patient-specific value proposal module P4 includes a test toxin concentration processing module P41, a predicted toxin concentration processing module P42, and a patient-specific value post-processing module P43. The function implemented by executing the test toxin concentration processing module P41 is the same as the function of the test toxin concentration processing unit 41. The function implemented by executing the predicted toxin concentration processing module P42 is the same as the function of the predicted toxin concentration processing unit 42. The function implemented by executing the patient-specific value post-processing module P43 is the same as the function of the patient-specific value post-processing unit 43.

The predicted toxin concentration processing module P42 includes a patient-specific value pre-processing module P421 and a toxin concentration calculation module P422. The function implemented by executing the patient-specific value pre-processing module P421 is the same as the function of the patient-specific value pre-processing unit 421. The function implemented by executing the toxin concentration calculation module P422 is the same as the function of the toxin concentration calculation unit 422.

The patient-specific value pre-processing module P421 includes a patient-specific value receiving module P421a, a patient test value receiving module P421b, a dialysis conditional value receiving module P421c, and a coefficient calculation module P421d. The function implemented by executing the patient-specific value receiving module P421a is the same as the function of the patient-specific value receiving unit 421a. The function implemented by executing the patient test value receiving module P421b is the same as the function of the patient test value receiving unit 421b. The function implemented by executing the dialysis conditional value receiving module P421c is the same as the function of the dialysis conditional value receiving unit 421c. The function implemented by executing the coefficient calculation module P421d is the same as the function of the coefficient calculation unit 421d.

The toxin concentration calculation module P422 includes a during-dialysis extracellular fluid calculation module P422a, a during-dialysis intracellular fluid calculation module P422b, a post-dialysis extracellular fluid calculation module P422c, and a post-dialysis intracellular fluid calculation module P422d. The function implemented by executing the during-dialysis extracellular fluid calculation module P422a is the same as the function of the during-dialysis extracellular fluid calculation unit 422a. The function implemented by executing the during-dialysis intracellular fluid calculation module P422b is the same as the function of the during-dialysis intracellular fluid calculation unit 422b. The function implemented by executing the post-dialysis extracellular fluid calculation module P422c is the same as the function of the post-dialysis extracellular fluid calculation unit 422c. The function implemented by executing the post-dialysis intracellular fluid calculation module P422d is the same as the function of the post-dialysis intracellular fluid calculation unit 422d.

The search dialysis conditional value proposal module P5 includes a dialysis conditional value pre-processing module P51, a dialysis conditional value acquisition module P52, and a toxin concentration calculation module P53. The function implemented by executing the dialysis conditional value pre-processing module P51 is the same as the function of the dialysis conditional value pre-processing unit 51. The function implemented by executing the dialysis conditional value acquisition module P52 is the same as the function of the search dialysis conditional value acquisition unit 52. The function implemented by executing the toxin concentration calculation module P53 is the same as the function of the toxin concentration calculation unit 53.

The dialysis conditional value pre-processing module P51 includes a patient-specific value receiving module P511, a patient test value receiving module P512, a target dialysis conditional value receiving module P513, a known dialysis conditional value receiving module P514, and a coefficient calculation module P515. The function implemented by executing the patient-specific value receiving module P511 is the same as the function of the patient-specific value receiving unit 511. The function implemented by executing the patient test value receiving module P512 is the same as the function of the patient test value receiving unit 512. The function implemented by executing the target dialysis conditional value receiving module P513 is the same as the function of the target dialysis conditional value receiving unit 513. The function implemented by executing the known dialysis conditional value receiving module P514 is the same as the function of the known dialysis conditional value receiving unit 514. The function implemented by executing the coefficient calculation module P515 is the same as the function of the coefficient calculation unit 515.

The dialysis conditional value acquisition module P52 includes a condition setting module P521 and a determination module P522. The function implemented by executing the condition setting module P521 is the same as the function of the condition setting unit 521. The function implemented by executing the determination module P522 is the same as the function of the determination unit 522.

The toxin concentration calculation module P53 includes a during-dialysis extracellular fluid calculation module P531, a during-dialysis intracellular fluid calculation module P532, a post-dialysis extracellular fluid calculation module P533, and a post-dialysis intracellular fluid calculation module P534. The function implemented by executing the during-dialysis extracellular fluid calculation module P531 is the same as the function of the during-dialysis extracellular fluid calculation unit 531. The function implemented by executing the during-dialysis intracellular fluid calculation module P532 is the same as the function of the during-dialysis intracellular fluid calculation unit 532. The function implemented by executing the post-dialysis extracellular fluid calculation module P533 is the same as the function of the post-dialysis extracellular fluid calculation unit 533. The function implemented by executing the post-dialysis intracellular fluid calculation module P534 is the same as the function of the post-dialysis intracellular fluid calculation unit 534.

### Execution Example

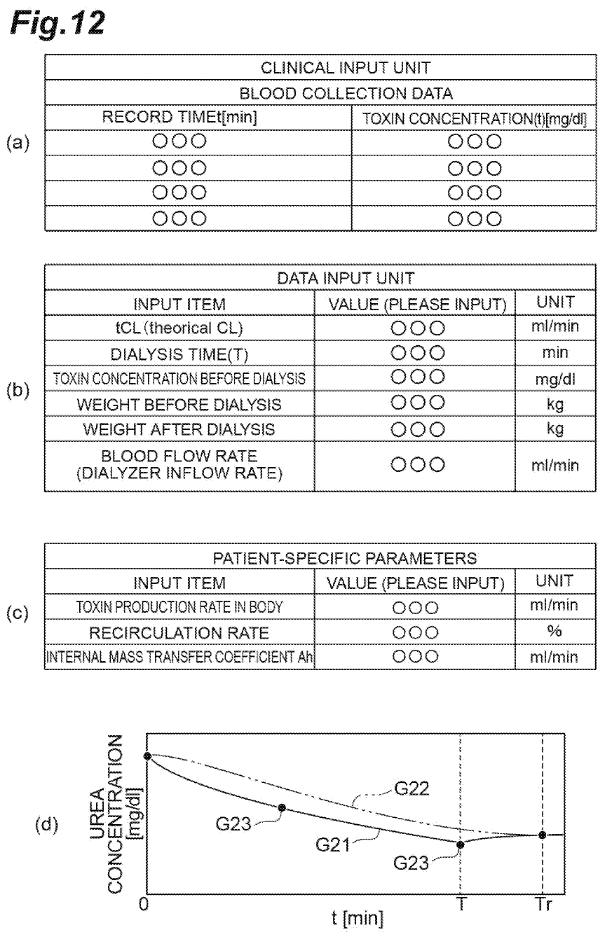

A mode shown on the display unit 3 when the dialysis information providing program P1 is executed will be exemplified. FIGS. 12(a), 12(b), 12(c), and 12(d) illustrate modes shown on the display unit 3 when the patient-specific value proposal module P4 is executed in the dialysis information providing program P1.

FIG. 12(a) illustrates an execution result of the test toxin concentration processing module P41. That is, the table illustrated in in FIG. 12(a) corresponds to the test toxin concentration processing unit 41. FIG. 12(b) illustrates an execution result of the patient test value receiving module P421b and the dialysis conditional value receiving module P421c. That is, the table illustrated in FIG. 12(b) corresponds to the patient test value receiving unit 421b and the dialysis conditional value receiving unit 421c. FIG. 12(c) illustrates an execution result of the patient-specific value receiving module P421a. That is, the table illustrated in FIG. 12(c) corresponds to the patient-specific value receiving unit 421a. FIG. 12(d) is an example of information displayed by the display unit 3 that has received the data θ6. FIG. 12(d) illustrates predicted toxin concentrations (graphs G21, G22) and a test toxin concentration G23.

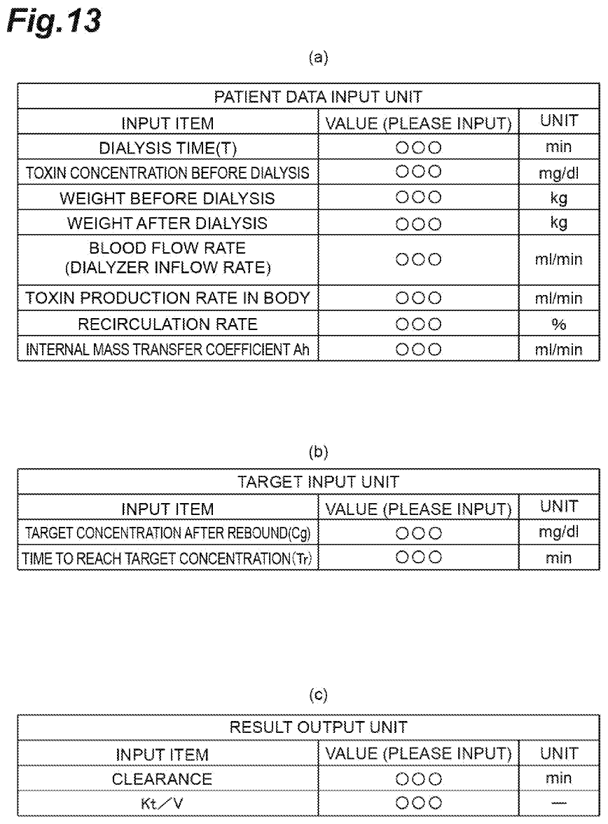

Another mode shown on the display unit 3 when the dialysis information providing program P1 is executed will be exemplified. FIGS. 13(a), 13(b), and 13(c) illustrate modes shown on the display unit 3 when the search dialysis conditional value proposal module P5 is executed in the dialysis information providing program P1.

FIG. 13(a) illustrates an execution result of the patient-specific value receiving module P511, the patient test value receiving module P512, and the known dialysis conditional value receiving module P514. That is, the table illustrated in FIG. 13(a) corresponds to the patient-specific value receiving unit 511, the patient test value receiving unit 512, and the known dialysis conditional value receiving unit 514. FIG. 13(b) illustrates an execution result of the target dialysis conditional value receiving module P513. That is, the table illustrated in FIG. 13(b) corresponds to the target dialysis conditional value receiving unit 513. FIG. 13(c) is an example of information displayed by the display unit 3 that has received the data φ5. FIG. 13(c) illustrates a proposed clearance value and a dialysis index (Kt/V) obtained from the clearance value.

### <Modification>

The embodiments of the present invention have been described. The present invention is not limited to the embodiments descried above. The present invention may be modified without changing the gist described in each claim. Further, the present invention may include additional constituent elements without changing the gist described in each claim.

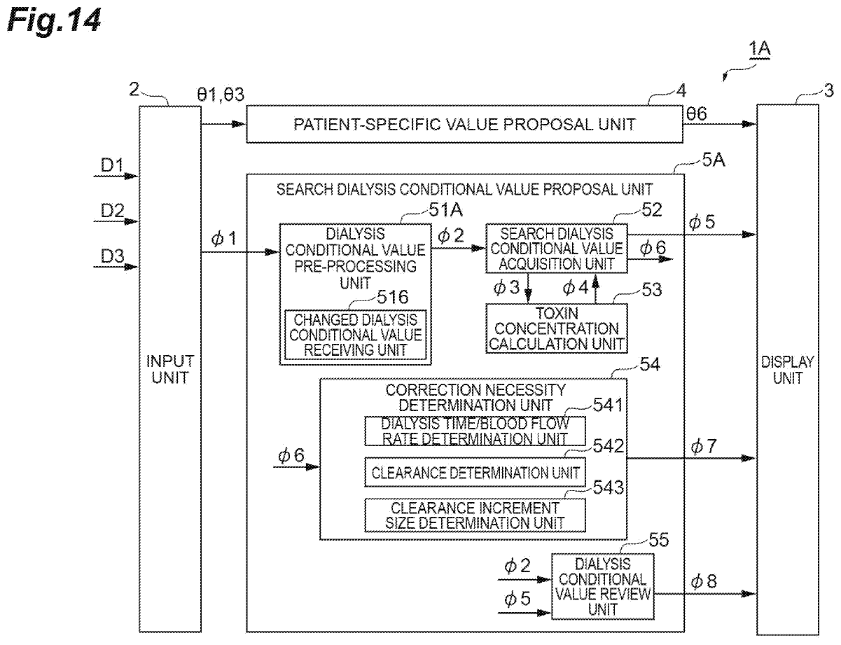

FIG. 14 is a functional block diagram illustrating a dialysis information providing device 1A according to a modification. The dialysis information providing device 1A may have a function of evaluating an input numerical value. Further, the dialysis information providing device 1A may have a function for reviewing other dialysis conditions based on a search dialysis conditional value obtained.

A search dialysis conditional value proposal unit 5A includes a dialysis conditional value pre-processing unit 51A, the search dialysis conditional value acquisition unit 52, the toxin concentration calculation unit 53, a correction necessity determination unit 54, and a dialysis conditional value review unit 55. That is, the search dialysis conditional value proposal unit 5A additionally includes the correction necessity determination unit 54 and the dialysis conditional value review unit 55 as compared with the search dialysis conditional value proposal unit 5 of the embodiment illustrated in FIG. 9.

### <Correction Necessity Determination Unit>

The correction necessity determination unit 54 has a function of evaluating an input numerical value. The dialysis conditional value review unit 55 has a function for reviewing other dialysis conditions based on a search dialysis conditional value obtained.

The correction necessity determination unit 54 receives data φ6 from the search dialysis conditional value acquisition unit 52. The data φ6 includes one or a plurality of determination results generated by the determination unit 522. The determination unit 522 determines whether data φ4 (predicted toxin concentration value) returned from the toxin concentration calculation unit 53 as a result of passing data φ3 to the toxin concentration calculation unit 53 satisfies data φ23 (target dialysis conditional value). For example, in a case where the predicted toxin concentration value is greater than the target dialysis conditional value, the determination unit 522 adds a sign “−1” to the search dialysis conditional value (clearance). In a case where the predicted toxin concentration value satisfies the target dialysis conditional value, the determination unit 522 adds a sign “+1” to the search dialysis conditional value (clearance). In a case where the predicted toxin concentration value is less than the target dialysis conditional value, the determination unit 522 adds a sign “O” to the search dialysis conditional value (clearance). Note that the content of the signs is an example, and thus may be appropriately changed. Therefore, the data 96 includes one or a plurality of sets including the clearance value and the sign.

The correction necessity determination unit 54 uses the data φ6 to generate data φ7 indicating a result of evaluating the input numerical value. The correction necessity determination unit 54 passes the data φ7 to the display unit 3. The display unit 3 displays the data φ7.

The correction necessity determination unit 54 includes one or a plurality of determination units according to dialysis conditions to be determined. For example, the correction necessity determination unit 54 of the modification includes a dialysis time/blood flow rate determination unit 541, a clearance determination unit 542, and a clearance increment size determination unit 543.

The dialysis time/blood flow rate determination unit 541 determines whether the values of the dialysis time and the blood flow rate are appropriate.

The dialysis time/blood flow rate determination unit 541 counts, as for a plurality of signs included in the data θ6, the number of signs (−1) indicating that the predicted toxin concentration value is greater than the target toxin concentration value. The dialysis time/blood flow rate determination unit 541 determines whether the number of signs (−1) is greater than 0.

First, when the number of signs (−1) is greater than 0, the dialysis time/blood flow rate determination unit 541 performs computation of subtracting the value of the clearance from the blood flow rate (dialyzer inflow value). The dialysis time/blood flow rate determination unit 541 determines whether the result of the computation is greater than 0. When the computation result is greater than 0, the dialysis time/blood flow rate determination unit 541 outputs information indicating that the values of the dialysis time and the blood flow rate are appropriate. When the computation result is not greater than 0, the dialysis time/blood flow rate determination unit 541 outputs information to prompt a change in the values of the dialysis time and the blood flow rate.

On the other hand, when the number of signs (−1) is not greater than 0, the dialysis time/blood flow rate determination unit 541 outputs information to prompt a change in the values of the dialysis time and the blood flow rate.

The dialysis time/blood flow rate determination unit 541 generates data φ7. The data φ7 includes any one of the information indicating that the values of the dialysis time and the blood flow rate are appropriate and the information to prompt a change in the values of the dialysis time and the blood flow rate. For example, when the data φ7 includes the information indicating that the values of the dialysis time and the blood flow rate are appropriate, the display unit 3 that has received the data φ7 displays “OK”. For example, when the data φ7 includes the information to prompt a change in the values of the dialysis time and the blood flow rate, the display unit 3 that has received the data φ7 displays “Please change the dialysis time or the blood flow rate”. More specifically, “Please increase the dialysis time or the blood flow rate” or “Please reduce the dialysis time or the blood flow rate” may be displayed.

The clearance determination unit 542 determines whether the value of the clearance is appropriate. The clearance determination unit 542 herein is an initial value of the clearance that is a search dialysis conditional value. Depending on the initial value of the clearance, even when the calculation is repeated by changing the clearance, the data φ4 (predicted toxin concentration) satisfying data φ22 (target toxin concentration) may not be obtained. The clearance determination unit 542 determines whether the initial value of the clearance is appropriate.

The clearance determination unit 542 obtains the sign included in the first combination among the plurality of combinations of the clearance and the sign included in the data φ6. In a case where the sign included in the first combination indicates that the predicted toxin concentration value is greater than the target toxin concentration value (−1), information to prompt a change in the initial value of the clearance is output. For example, the clearance determination unit 542 may output information to prompt a reduction in the initial value of the clearance.

The clearance determination unit 542 obtains the sign included in the last combination among the plurality of combinations of the clearance and the sign included in the data φ6. In a case where the sign included in the last combination indicates that the predicted toxin concentration value is less than the target toxin concentration value (0), information to prompt a change in the initial value of the clearance is output. For example, the clearance determination unit 542 may output information to prompt an increase in the initial value of the clearance.

The clearance determination unit 542 determines whether there is a sign (+1) indicating that the predicted toxin concentration value satisfies the target toxin concentration value among the plurality of combinations of the clearance and the sign included in the data φ6. In a case where there is a sign (+1) indicating that the predicted toxin concentration value satisfies the target toxin concentration value, the clearance determination unit 542 outputs information indicating that the value of the clearance is appropriate.

The clearance determination unit generates data θ7. The data φ7 includes any one of the information indicating that the value of the clearance is appropriate and the information to prompt a change in the value of the clearance. For example, when the data φ7 includes the information indicating that the value of the clearance is appropriate, the display unit 3 that has received the data φ7 displays “OK”. For example, when the data φ7 includes the information to prompt a change in the value of the clearance, the display unit 3 that has received the data φ7 displays “Please change the initial value of the clearance”. The display unit 3 may perform display to prompt a change in other dialysis conditions that influence the value of the clearance. For example, the display unit 3 may display “Please reduce the initial value of the clearance or shorten the dialysis time”. For example, the display unit 3 may display “If a recirculation rate is set, please increase the initial value of the clearance”.

The clearance increment size determination unit 543 determines whether the increment size of the clearance is appropriate. The search dialysis conditional value acquisition unit 52 passes data φ3 including the clearance to the toxin concentration calculation unit 53. As a result, the toxin concentration calculation unit 53 returns the data 94 (predicted toxin concentration) to the search dialysis conditional value acquisition unit 52. The search dialysis conditional value acquisition unit 52 changes the value of the clearance in a case where the data φ4 (predicted toxin concentration) does not satisfy the data φ22 (target toxin concentration). When the value of the clearance is changed, the value to be added or subtracted is the clearance increment size. For example, in a case where the clearance increment size is excessively large, even when the calculation is repeated by changing the clearance, the data φ4 (predicted toxin concentration) satisfying the data φ22 (target toxin concentration) may not be obtained. In view of this, the clearance increment size determination unit 543 determines whether the increment size of the clearance is appropriate.

The clearance increment size determination unit 543 searches for the sign (+1) indicating that the predicted toxin concentration value satisfies the target toxin concentration value. The clearance increment size determination unit 543 then obtains a value of the clearance correlated with the sign (+1) indicating that the predicted toxin concentration value satisfies the target toxin concentration value. The clearance increment size determination unit 543 searches for the sign (0) indicating that the predicted toxin concentration value is less than the target toxin concentration value. The sign (0) searched here is the one immediately after switching from the sign (+1). The clearance increment size determination unit 543 then obtains a value of the clearance correlated with the sign (0) indicating that the predicted toxin concentration value satisfies the target toxin concentration value. The clearance increment size determination unit 543 performs computation of subtracting the value of the clearance correlated with the sign (0) from the value of the clearance correlated with the sign (+1). The clearance increment size determination unit 543 determines whether the result of the computation is greater than 0. When the computation result is greater than 0, the clearance increment size determination unit 543 outputs information to prompt a change in the increment size of the clearance. When the computation result is not greater than 0, the clearance increment size determination unit 543 outputs information indicating that the clearance increment size is appropriate.

The clearance increment size determination unit 543 generates data φ7. The data φ7 includes any one of the information indicating that the increment size of the clearance is appropriate and the information to prompt a change in the increment size of the clearance. For example, when the data φ7 includes the information indicating that the increment size of the clearance is appropriate, the display unit 3 that has received the data φ7 displays “OK”. For example, when the data φ7 includes the information to prompt a change in the increment size of the clearance, the display unit 3 that has received the data φ7 displays “Please change the initial value of the clearance”. The display unit 3 may perform display to prompt a change in other dialysis conditions that influence the value of the clearance. For example, the display unit 3 may display “Please change the increment size of the clearance”. More specifically, the display unit 3 may display “Please reduce the increment size of the clearance”.

The correction necessity determination unit 54 generates the data 97. The correction necessity determination unit 54 then passes the data φ7 to the display unit 3.

### <Dialysis Conditional Value Review Unit>

The dialysis conditional value review unit 55 uses a search dialysis conditional value that satisfies the target dialysis conditional value to evaluate whether the numerical value of a dialysis conditional value different from the search dialysis conditional value can be changed. For example, the dialysis conditional value review unit 55 evaluates whether it is possible to shorten the dialysis time while maintaining a dialysis index value (Kt/V) obtained as the search dialysis conditional value that satisfies the target dialysis conditional value.

The dialysis conditional value review unit 55 receives data φ5 from the search dialysis conditional value acquisition unit 52. The dialysis conditional value review unit 55 receives data φ2 from a changed dialysis conditional value receiving unit 516 of the dialysis conditional value pre-processing unit 51A. The dialysis conditional value review unit 55 uses the data φ2 and φ5 to generate data φ8. The dialysis conditional value review unit 55 passes the data φ7 to the display unit 3.

Specifically, the dialysis conditional value review unit 55 obtains the dialysis index value (Kt/V) by using the clearance value satisfying the target toxin concentration included in the data φ5. Further, the dialysis conditional value review unit 55 obtains a changed clearance value included in φ2 as the changed dialysis conditional value. The dialysis conditional value review unit 55 then uses the dialysis index value (Kt/V), the changed clearance value, and the total body water before dialysis to obtain a changed dialysis time (T′).

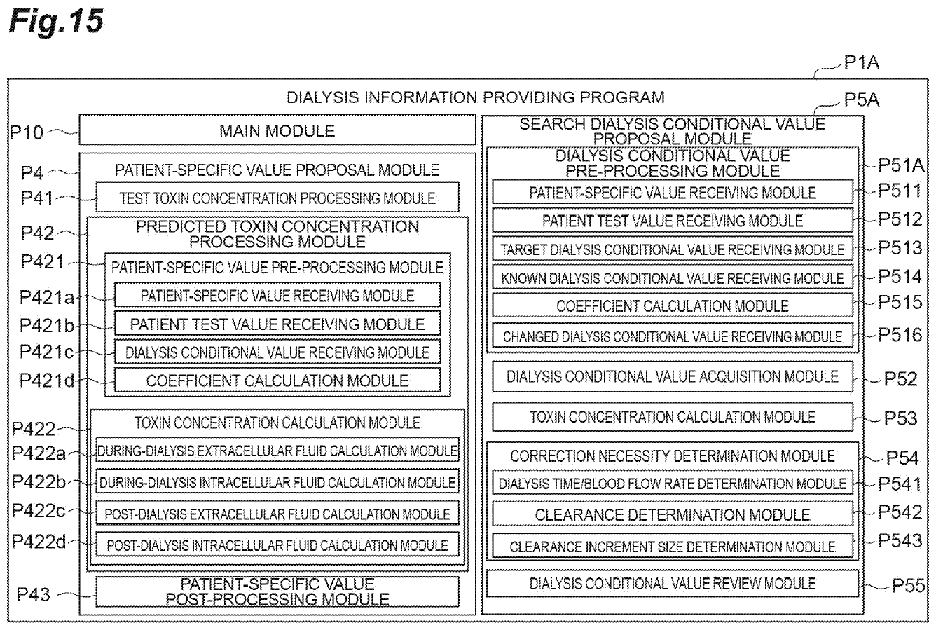

As illustrated in FIG. 15, a dialysis information providing program PIA includes the main module P10, the patient-specific value proposal module P4, and a search dialysis conditional value proposal module P5A.

Since the main module P10 and the patient-specific value proposal module P4 are the same as those in the embodiment, detailed description thereof is omitted.

The search dialysis conditional value proposal module P5A includes a dialysis conditional value pre-processing module P51A, the dialysis conditional value acquisition module P52, the toxin concentration calculation module P53, a correction necessity determination module P54, and a dialysis conditional value review module P55. The function implemented by executing the dialysis conditional value pre-processing module P51A is the same as the function of the dialysis conditional value pre-processing unit 51A. The function implemented by executing the correction necessity determination module P54 is the same as the function of the correction necessity determination unit 54. The function implemented by executing the dialysis conditional value review module P55 is the same as the function of the dialysis conditional value review unit 55.

The correction necessity determination module P54 includes a dialysis time/blood flow rate determination module P541, a clearance determination module P542, and a clearance increment size determination module P543. The function implemented by executing the dialysis time/blood flow rate determination module P541 is the same as the function of the dialysis time/blood flow rate determination unit 541. The function implemented by executing the clearance determination module P542 is the same as the function of the clearance determination unit 542. The function implemented by executing the clearance increment size determination module P543 is the same as the function of the clearance increment size determination unit 543.

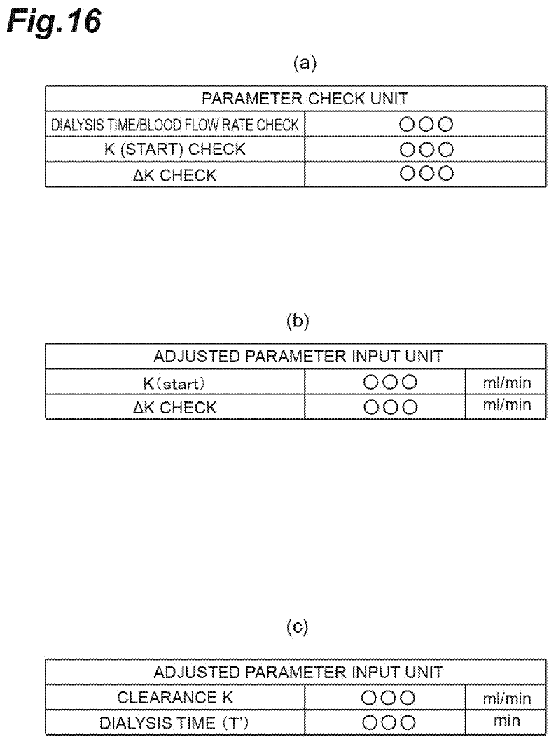

The execution result of the dialysis information providing program PIA may include those illustrated in FIGS. 16(a), 16(b), and 16(c) in addition to those illustrated in FIG. 13. FIG. 16(a) is an example of screen display showing an execution result of the correction necessity determination unit 54. FIG. 16(b) is an example of screen display with which to input the initial value of the clearance and the clearance increment size. FIG. 16(c) is an example of screen display with which to input the clearance value designated in the dialysis conditional value review unit 55 and to present the recalculated dialysis time.

### Usage Example

Some of use cases of the dialysis information providing devices 1 and 1A according to the modification will be described. Note that the following use cases are examples.

For example, in a case where a clearance value is proposed as the search dialysis conditional value, dialysis conditions different from the clearance may be reviewed. The use of the clearance determines the dialysis index value (Kt/V). Thus, the dialysis index value (Kt/V) may also be treated substantially as a value proposed as the search dialysis conditional value.

For example, a dialyzer is designated in some cases. The designation of the dialyzer means that the value of the clearance is a fixed value. In such a case, the dialysis conditional value review unit 55 is used. The changed dialysis conditional value receiving unit 516 receives a clearance value corresponding to the designated dialyzer as the changed dialysis conditional value. The changed dialysis conditional value receiving unit 516 uses the proposed dialysis index (Kt/V) and the received clearance to calculate a dialysis time as the proposed dialysis index (Kt/V).

Another use case is review of the dialysis time and/or the blood flow rate. It is desirable to adjust the dialysis time and/or blood flow rate for each condition of the patient. In such a case, the correction necessity determination unit 54 is used. A new dialysis time and/or blood flow rate is input to the dialysis information providing device 1A. Then, the correction necessity determination unit 54 determines the validity of the computation based on the reinput dialysis time and/or blood flow rate. For example, if “OK” is displayed on the display unit 3 as a result of the input of the new value for the dialysis time and/or blood flow rate, it is informed that the new value can be used for the dialysis time and/or blood flow rate. Further, as a result of the input of the new value for the dialysis time and/or blood flow rate, a dialysis index (Kt/V) corresponding to the new value of the dialysis time and/or blood flow rate is displayed on the display unit 3. The dialysis index (Kt/V) can be used to examine the validity of the new value of the dialysis time and/or blood flow rate.

As a more specific use case, a study on how much the blood flow rate can be reduced from the viewpoint of reducing the physical burden on the patient is exemplified. In this case, various blood flow rates are input to the dialysis information providing device 1A. This obtains a dialysis index (Kt/V); therefore, the blood flow rate can be determined while examining the dialysis index (Kt/V). It is further possible to try to make adjustment together with other dialysis conditions such as a dialysis time.

In short, what can be implemented by the dialysis information providing devices 1 and 1A is listed as follows. First, in the dialysis information providing devices 1 and 1A, the toxin concentration calculation unit 53 can accurately reproduce the temporal transition of concentration of urea nitrogen in consideration of the rebound phenomenon and the recirculation caused by the vascular access. Further, since the temporal transition of concentration of urea nitrogen can be accurately reproduced, it is possible to obtain a patient-specific value by utilizing the test value. Since the patient-specific value can be obtained for each patient, the mass transfer characteristics in the body of a patient can be well grasped. The dialysis information providing devices 1 and 1A can quantitatively evaluate the recirculation caused by the vascular access. The dialysis information providing devices 1 and 1A can obtain a dialysis index (Kt/V) in consideration of the recirculation.

According to some of the functional effects described above, the dialysis information providing devices 1 and 1A can accurately predict the temporal transition of concentration of urea nitrogen by taking account of influence of the rebound phenomenon and the recirculation. Further, the dialysis information providing devices 1 and 1A can satisfactorily grasp the mass transfer characteristics in the body of a patient. The dialysis information providing devices 1 and 1A then can propose dialysis conditions required to achieve the set dialysis target. Examples of the dialysis that can be proposed include clearance, a dialysis time, a blood flow rate, water removal, a dialysate volume, and a dialysis index (Kt/V).

The dialysis information providing devices 1 and 1A use Formulas (1) to (4) derived from a two-compartment model described later. Formulas used by the dialysis information providing devices 1 and 1A to predict a toxin concentration are not limited to Formulas (1) to (4). The dialysis information providing devices 1 and 1A may appropriately use a function that can predict a toxin concentration. For example, the dialysis information providing devices 1 and 1A may use a formula derived from a one-compartment model described later.

The dialysis information providing devices 1 and 1A can be applied to several dialysis methods in addition to normal dialysis.

The dialysis information providing devices 1 and 1A can also assist in determining dialysis conditions for hemodialysis (HD), overnight dialysis, hemodiafiltration (HF), intermittent infusion hemodiafiltration (I-HDF), on-line hemodiafiltration (on-line HDF), and off-line hemodiafiltration (off-line HDF). Note that ultrafiltration (ECUM) may be performed at any time of the dialysis treatments.

In the case of hemodialysis (HF), a volume of fluid replacement and an ultrafiltration volume are added as necessary dialysis conditions. In the case of on-line hemodiafiltration (on-line HDF) and off-line hemodiafiltration (off-line HDF), a volume of fluid replacement and an ultrafiltration volume are added as necessary dialysis conditions. In the case of intermittent infusion hemodiafiltration (I-HDF), a total volume of fluid replacement and a fluid replacement interval are added as necessary dialysis conditions. Further, in the case of intermittent infusion hemodiafiltration (I-HDF), a fluid replacement rate and the number of times of refilling are added as necessary dialysis conditions. The volume of fluid replacement and the ultrafiltration volume can also be given as an argument or a return value. The volume of fluid replacement and the ultrafiltration volume can also be determined from clearance obtained as the calculation result using a catalog.

In the above embodiments, the configuration for presenting clearance has been exemplified. That is, one condition is selected as the search dialysis condition from among a plurality of dialysis conditions. As initially described, the dialysis information providing devices 1 and 1A can provide useful information for determining two or more conditions selected from among a plurality of dialysis conditions. For example, regarding two types of parameters of the dialysis conditions, the dialysis information providing devices 1 and 1A can show, in a graph, a relationship to be satisfied in order to achieve the dialysis target. Even when there are some restrictions on the dialysis conditions that can be set, a user of the dialysis information providing devices 1 and 1A can examine the dialysis conditions based on the graph.

In the above embodiments, the configuration in which the search dialysis condition is clearance and the known dialysis conditions are a dialysis time and a blood flow rate is exemplified. Some other configurations will be listed. As a first example, an example can be taken in which the search dialysis condition is a dialysis time and the known dialysis conditions are clearance and the amount of blood. As a second example, an example can be taken in which the search dialysis condition is a blood flow rate and the known dialysis conditions are a dialysis time and clearance. As a third example, an example can be taken in which the search dialysis conditions are a dialysis time and clearance, and the known dialysis condition is a blood flow rate. As a fourth example, an example can be taken in which the search dialysis conditions are clearance and a blood flow rate and the known dialysis condition is a dialysis time. As a fifth example, an example can be taken in which the search dialysis conditions are a dialysis time and a blood flow rate and the known dialysis condition is clearance.

<Prediction of Transition of Toxin Amount in Living Body During and after Dialysis>

In order to ensure that a patient undergoing dialysis has a good prognosis in the long term, increasing the treatment efficiency is important. In addition, the condition of the patient also needs to be carefully managed. The condition of the patient means the transition of the amount of toxins present in the body of the patient. In view of this, the inventors have intensively studied a technique for accurately predicting the transition of the amount of toxins present in the body of a patient during and after dialysis.

The inventors employed two models as a model for predicting the transition of the amount of toxins. Then, formulas for predicting the transition of the amount of toxins were derived based on each model. A first model is a one-compartment model. The one-compartment model is a model widely used in clinical practice at present. A second model is a two-compartment model.

### <Prediction Formula Based on One-Compartment Model>

The one-compartment model simulates a patient to be dialyzed as one compartment. In this case, it can be assumed that the concentration of the substance is uniform in the entire body of the patient. It can also be assumed that the transition of the concentration of the substance over time is uniform in the entire body of the patient.

As a representative example of the prediction formula for the transition of the toxin amount based on such assumptions, a theoretical formula shown in Formula (5) can be given. Formula (5) defines the relationship between the amount of toxins removed by dialysis and the amount of toxins that decrease inside the living body. The first term on the right side of Formula (5) represents the amount of toxins removed per unit time by dialysis. The second term on the right side of Formula (5) represents the amount of toxins produced per unit time in the living body.

$$\begin{matrix}[ Formula 9 ] & \\ {V}_{bo} \frac{{dC}_{bo} ( t )}{dt} = - CL \cdot {C}_{bo} ( t ) + S & ( 5 )\end{matrix}$$

V_{bo}: body fluid volume
C_{bo}(t): toxin concentration in living body at time (t)
CL: clearance
S: amount of toxins produced per unit time in living body

Formula (5) is integrated to yield Formula (6). Formula (6) represents the toxin concentration during dialysis. Formula (6) is a function in which time (t) is an independent variable and the toxin concentration is a dependent variable (objective variable). By substituting the numerical value of each variable into the right side of Formula (6), the toxin concentration at time (t) is obtained.

$$\begin{matrix}[ Formula 10 ] & \\ {C}_{bo} ( t ) = {I}_{1} exp ( - \frac{CL}{{V}_{bo}} t ) + \frac{S}{CL} & ( 6 )\end{matrix}$$

$$\begin{matrix}[ Formula 11 ] & \\ {I}_{1} = {C}_{s} - \frac{S}{CL} & ( 7 )\end{matrix}$$

I₁: constant of integration
Cs: initial concentration at t=0

After dialysis (t≥T), blood purification is complete. That is, CL=0 [ml/min]. Under this condition, Formula (5) is integrated with time (t) to yield Formula (8). Formula (8) represents the toxin concentration after dialysis. As with Formula (6), Formula (8) is a function in which time (t) is an independent variable and the toxin concentration is a dependent variable (objective variable). By substituting the numerical value of each variable into the right side of Formula (8), the toxin concentration at time (t) is obtained.

$$\begin{matrix}[ Formula 12 ] & \\ {C}_{bo} ( t ) = {I}_{2} + \frac{S}{{V}_{bo}} t & ( 8 )\end{matrix}$$

$$\begin{matrix}[ Formula 13 ] & \\ {I}_{2} = ( {C}_{s} - \frac{S}{CL} ) exp ( - \frac{CL}{{V}_{bo}} T ) + \frac{S}{CL} - \frac{S}{{V}_{bo}} T & ( 9 )\end{matrix}$$

I₂: constant of integration

### <Prediction Formula Based on Two-Compartment Model>

The two-compartment model simulates a patient to be dialyzed as two compartments. The body fluid in the living body includes extracellular fluid and intracellular fluid. The extracellular fluid includes plasma and interstitial fluid. The interstitial fluid is a liquid present in a gap between cells. There is a cell wall between the intracellular fluid and the interstitial fluid. The cell wall acts to prevent substances from moving. The action of preventing substances from moving is referred to as a mass transfer resistance. Further, there is a blood vessel wall between the interstitial fluid and the plasma. The blood vessel wall also has a mass transfer resistance that is an action of preventing substances from moving. The mass transfer resistance of the blood vessel wall is sufficiently smaller than the mass transfer resistance of the cell wall. That is, uneven toxin concentration between the intracellular fluid and the extracellular fluid occurs mainly across the cell wall. Accordingly, in the two-compartment model, the first compartment is defined as the extracellular fluid, and the second compartment is defined as the intracellular fluid.

During dialysis (0≤t≤T), water and toxins contained in the extracellular fluid are removed by the dialysis machine. The extracellular fluid herein is blood. Further, between the intracellular fluid and the extracellular fluid, toxins transfer from the intracellular fluid to the extracellular fluid. The transfer of toxins is based on diffusion that occurs due to differences in toxin concentration. Further, the transfer of toxins is based on the transfer of water from the intracellular fluid to the extracellular fluid. The transfer of water from the intracellular fluid to the extracellular fluid is referred to as plasma refilling.

The balance between the amount of toxins contained in the extracellular fluid and the amount of toxins contained in the intracellular fluid is represented in Formulas (10) and (11).

$$\begin{matrix}[ Formula 14 ] & \\ \frac{{dV}_{ex} ( t ) {C}_{ex} ( t )}{dt} = - CL \cdot {C}_{ex} ( t ) - Ah [ {C}_{ex} ( t ) - {C}_{in} ( t ) ] + {ω}_{pl} {C}_{in} ( t ) & ( 10 )\end{matrix}$$

$$\begin{matrix}[ Formula 15 ] & \\ \frac{{dV}_{in} ( t ) {C}_{in} ( t )}{dt} = Ah [ {C}_{ex} ( t ) - {C}_{in} ( t ) ] - {ω}_{pl} {C}_{in} ( t ) + S & ( 11 )\end{matrix}$$

Here, Vₑₓ(t) and Vᵢₙ(t) represent the extracellular fluid volume and the intracellular fluid volume at time (t), Cₑₓ(t) and Cᵢₙ(t) represent the toxin concentrations in the extracellular fluid and the intracellular fluid at time (t), Ah represents the overall mass transfer coefficient in the cell wall, and ωₚₗ represents the plasma refilling rate.

Formula (10) relates to the extracellular fluid. The left side of Formula (10) represents a temporal change in the amount of toxins contained in the extracellular fluid per unit volume in consideration of a change in volume due to ultrafiltration in the dialysis machine. The first term on the right side of Formula (10) represents the amount of toxins removed per unit time by dialysis. The second term on the right side of Formula (10) represents the amount of toxins that migrate in the cell wall by diffusion. The third term on the right side of Formula (10) represents the amount of toxins transferred by plasma refilling.

Formula (11) relates to the intracellular fluid. The left side of Formula (11) represents a temporal change in the amount of toxins contained in the intracellular fluid per unit volume. In the left side of Formula (11), a change in volume due to ultrafiltration in the dialysis machine is taken into consideration. The first term on the right side of Formula (11) represents the amount of toxins that migrate in the cell wall by diffusion. The second term on the right side of Formula (11) represents the amount of toxins transferred by plasma refilling. The third term on the right side of Formula (11) represents the amount of toxins produced per unit time in a cell.

It is assumed that the fluid volume of the extracellular fluid and the fluid volume of the intracellular fluid change over time due to water removal by dialysis and plasma refilling. According to this assumption, the liquid volume of the extracellular fluid is represented by Formula (12). The liquid volume of the intracellular liquid is represented by Formula (13).

$$\begin{matrix}[ Formula 16 ] & \\ {V}_{in} ( t ) = {V}_{in} ( 0 ) - {ω}_{pl} t & ( 13 )\end{matrix}$$

$$\begin{matrix}[ Formula 17 ] & \\ {V}_{in} ( t ) = {V}_{in} ( 0 ) - {ω}_{pl} t & ( 13 )\end{matrix}$$

ω_{f}: water removal per unit time by dialysis

Further, as represented in Formula (14), it is assumed that the extracellular fluid volume and the intracellular fluid volume always maintain a constant ratio.

$$\begin{matrix}[ Formula 18 ] & \\ ε = \frac{{V}_{ex} ( t )}{{V}_{in} ( t )} = const . & ( 14 )\end{matrix}$$

From Formulas (12) to (14), the plasma filling amount ωₚₗ is represented by Formula (15). The plasma filling amount ωₚₗ may be treated as a constant. This is because the plasma filling amount ωₚₗ is almost a constant value when Vᵢₙ(0), Vₑₓ(0), ω_{f}, and ε in an actually possible range are substituted, regardless of the time (t).

$$\begin{matrix}[ Formula 19 ] & \\ {ω}_{pl} = \frac{ε {V}_{in} ( 0 ) - [ {V}_{ex} ( 0 ) - {ω}_{f} t ]}{( 1 + ) t} ≅ = const . & ( 15 )\end{matrix}$$

### <Derivation of Toxin Concentration Analytical Solution During Dialysis>

Formula (15) is applied to Formula (10) to yield Formula (16). Further, Formula (15) is applied to Formula (11) to yield Formula (17).

$$\begin{matrix}[ Formula 20 ] & \\ {V}_{ex} ( t ) \frac{{dC}_{ex} ( t )}{dt} = - CL \cdot {C}_{ex} ( t ) - Ah [ {C}_{ex} ( t ) - {C}_{in} ( t ) ] + {ω}_{pl} {C}_{in} ( t ) + ( {ω}_{f} - {ω}_{pl} ) {C}_{ex} ( t ) & ( 16 )\end{matrix}$$

$$\begin{matrix}[ Formula 21 ] & \\ {V}_{in} ( t ) \frac{{dC}_{in} ( t )}{dt} = Ah [ {C}_{ex} ( t ) - {C}_{in} ( t ) ] + S & ( 17 )\end{matrix}$$

During dialysis (0≤t≤T), the extracellular fluid volume (Vₑₓ) and the intracellular fluid volume (Vᵢₙ) change over time. Thus, it is impossible to directly derive a general solution of toxin concentration by using Formula (16) and Formula (17). Thus, time (t) is defined as a new variable. By introducing this definition, a function (Formula (1)) representing the toxin concentration in the extracellular fluid during dialysis can be obtained from Formula (16). Similarly, a function (Formula (2)) representing the toxin concentration in the intracellular fluid during dialysis can be obtained from Formula (17). Formulas (1) and (2) are functions with time (t) as a variable.

$$\begin{matrix}[ Formula 22 ] & \\ {C}_{ex} ( t ) = {{I}_{4} [ 1 - \frac{{ω}_{f} - {ω}_{pl}}{{V}_{ex} ( 0 )} t ]}^{\frac{{λ}_{1}}{{ω}_{f} - {ω}_{pl}}} + {{I}_{5} [ 1 - \frac{{ω}_{f} - {ω}_{pl}}{{V}_{ex} ( 0 )} t ]}^{\frac{{λ}_{2}}{{ω}_{f} - {ω}_{pl}}} + \frac{S}{γ} & ( 1 )\end{matrix}$$

$$\begin{matrix}[ Formula 23 ] & \\ {C}_{in} ( t ) = \frac{Ah + CL - {ω}_{f} + {ω}_{pl} - {λ}_{1}}{Ah + {ω}_{pl}} {{I}_{4} [ 1 - \frac{{ω}_{f} - {ω}_{pl}}{{V}_{ex} ( 0 )} t ]}^{\frac{{λ}_{1}}{{ω}_{f} - {ω}_{pl}}} + \frac{Ah + CL - {ω}_{f} + {ω}_{pl} - {λ}_{2}}{Ah + {ω}_{pl}} {{I}_{5} [ 1 - \frac{{ω}_{f} - {ω}_{pl}}{{V}_{ex} ( 0 )} t ]}^{\frac{{λ}_{2}}{{ω}_{f} - {ω}_{pl}}} + \frac{Ah + CL - {ω}_{f} + {ω}_{pl}}{Ah + {ω}_{pl}} \frac{S}{γ} & ( 2 )\end{matrix}$$

The variable (λ₁) and the variable (λ₂) included in Formulas (1) and (2) are represented by Formulas (18) to (21).

$$\begin{matrix}[ Formula 24 ] & \\ \begin{matrix}- {λ}_{1} \\ - {λ}_{2}\end{matrix} = \frac{- β ∓ \sqrt{{β}^{2} - 4 αγ}}{2 α} & ( 18 )\end{matrix}$$

$$\begin{matrix}[ Formula 25 ] & \\ α = \frac{1}{( Ah + {ω}_{pl} )} & ( 19 )\end{matrix}$$

$$\begin{matrix}[ Formula 26 ] & \\ β = \frac{( 1 + ε ) Ah + CL - {ω}_{f} + {ω}_{pl}}{ε ( Ah + {ω}_{pl} )} & ( 20 )\end{matrix}$$

$$\begin{matrix}[ Formula 27 ] & \\ γ = \frac{Ah ( CL - {ω}_{f} )}{Ah + {ω}_{pl}} & ( 21 )\end{matrix}$$

Formulas (1) and (2) include a constant of integration (I₄) and a constant of integration (I₅). The constant of integration (I₄) and the constant of integration (Is) can be obtained by giving initial conditions to Formulas (1) and (2). The initial conditions are represented by Formula (22) under the assumption that the toxin concentration at the start of dialysis is uniform in the entire body.

$$\begin{matrix}[ Formula 28 ] & \\ t = 0 \to {C}_{ex} ( 0 ) = {C}_{in} ( 0 ) = {C}_{s} & ( 22 )\end{matrix}$$

When the initial conditions represented in Formula (22) are given to Formula (1) and Formula (2), the constant of integration (I₄) represented in Formula (23) and the constant of integration (I₅) represented in Formula (24) are obtained.

$$\begin{matrix}[ Formula 29 ] & \\ {I}_{4} = \frac{Ah ( CL - {ω}_{f} ) ( - CL + {ω}_{f} + {λ}_{2} ) {C}_{s} - {λ}_{2} ( Ah + {ω}_{pl} ) S}{Ah ( CL - {ω}_{f} ) ( {λ}_{2} - {λ}_{1} )} & ( 23 )\end{matrix}$$

$$\begin{matrix}[ Formula 30 ] & \\ {I}_{5} = \frac{Ah ( CL - {ω}_{f} ) ( - CL + {ω}_{f} + {λ}_{1} ) {C}_{s} - {λ}_{1} ( Ah + {ω}_{pl} ) S}{Ah ( CL - {ω}_{f} ) ( {λ}_{2} - {λ}_{1} )} & ( 24 )\end{matrix}$$

The above Formulas (1) and (18) to (24) give a function in which time (t) indicating a temporal change in the toxin concentration of the extracellular fluid during dialysis is used as a variable. Determining the time (t) of this function gives the toxin concentration of the extracellular fluid during dialysis at the time (t).

Further, the above Formulas (2) and (18) to (24) give a function in which time (t) indicating a temporal change in the toxin concentration of the intracellular fluid during dialysis is used as a variable. Determining the time (t) of this function gives the toxin concentration of the intracellular fluid during dialysis at the time (t).

<Derivation of Toxin Concentration Analytical Solution after Dialysis>

A function indicating a temporal change in the toxin concentration after dialysis is derived from Formulas (10) and (11). After dialysis, the blood purification is completed. Thus, assuming that water is not ingested into the body, the conditions of CL=0 [ml/min] and ω_{f}=0 [ml/min] can be set. Further, since the transfer in the intracellular fluid also stops after dialysis, the condition of ωₚₗ=0 [ml/min] can also be set. That is, the extracellular fluid volume and the intracellular fluid volume after dialysis are constant in volume at time (t). Applying these conditions to Formula (10) yields Formula (25). Similarly, applying these conditions to Formula (11) yields Formula (26).

$$\begin{matrix}[ Formula 31 ] & \\ {V}_{ex} ( T ) = \frac{{dC}_{ex} ( t )}{dt} = - Ah [ {C}_{ex} ( t ) - {C}_{in} ( t ) ] & ( 25 )\end{matrix}$$

$$\begin{matrix}[ Formula 32 ] & \\ {V}_{in} ( T ) = \frac{{dC}_{in} ( t )}{dt} = Ah [ {C}_{ex} ( t ) - {C}_{in} ( t ) ] + S & ( 26 )\end{matrix}$$

After dialysis, the extracellular fluid volume is constant. Therefore, unlike Formula (16), Formula (25) can be used to obtain a general solution of the toxin concentration without introducing variable transformation. Similarly, unlike Formula (17), Formula (26) can be used to obtain a general solution of the toxin concentration without introducing variable transformation.

When Cᵢₙ(t) is eliminated by satisfying Formula (25) and Formula (26) simultaneously, a second-order linear differential equation (Formula (27)) related to Cₑₓ(t) is obtained.

$$\begin{matrix}[ Formula 33 ] & \\ \frac{{V}_{ex} ( T ) \cdot {V}_{in} ( T )}{Ah} \frac{{d}^{2} {C}_{ex} ( t )}{dt} + [ {V}_{ex} ( T ) + {V}_{in} ( T ) ] \frac{{dC}_{ex} ( t )}{dt} = S & ( 27 )\end{matrix}$$

A general solution to Formula (27) is Formula (3). Formula (3) is a function indicating a temporal change in the toxin concentration in the extracellular fluid after dialysis.

$$\begin{matrix}[ Formula 34 ] & \\ {C}_{ex} ( t ) = {I}_{6} exp ( - {λ}_{3} t ) + {I}_{7} + \frac{S}{{V}_{ex} ( t ) + {V}_{in} ( t )} t & ( 3 )\end{matrix}$$

Note that the coefficient (23) of Formula (3) is represented by Formula (28).

$$\begin{matrix}[ Formula 35 ] & \\ - {λ}_{3} = - \frac{Ah [ {V}_{ex} ( T ) + {V}_{in} ( T ) ]}{{V}_{ex} ( T ) \cdot {V}_{in} ( T )} & ( 28 )\end{matrix}$$

Formula (3) is substituted into Formula (25). This yields Formula (4) that is a general solution to Cᵢₙ(t). Formula (4) is a function indicating a temporal change in the toxin concentration in the intracellular fluid after dialysis.

$$\begin{matrix}[ Formula 36 ] & \\ {C}_{in} ( t ) = [ 1 - \frac{{V}_{ex} ( T )}{Ah} {λ}_{3} ] {I}_{6} exp ( - {λ}_{3} t ) + {I}_{7} + \frac{S}{{V}_{ex} ( t ) + {V}_{in} ( t )} [ t + \frac{{V}_{ex} ( T )}{Ah} ] & ( 4 )\end{matrix}$$

The constant of integration (I₆) and the constant of integration (I₇) are obtained by applying an intermediate condition to Formulas (1) and (2). As the intermediate condition, a function indicating the temporal change in the toxin concentration during dialysis represented in Formula (1) and Formula (2) is used. At the end of dialysis, that is, at t=T, the toxin concentrations (Cₑₓ(T)=C_{e,ex}. Cᵢₙ(T)=C_{e,in}) in the extracellular fluid and the intracellular fluid are calculated from Formulas (1) to (24). Then, the obtained toxin concentration is applied, as the intermediate condition, to Formulas (3) and (4). This yields the constant of integration (I₆) represented by Formula (29) and the constant of integration (I₇) represented by Formula (30).

$$\begin{matrix}[ Formula 37 ] & \\ {I}_{6} = - \frac{1}{{λ}_{3} exp ( - {λ}_{3} T )} [ \frac{( {C}_{e , in} - {C}_{e , ex} ) Ah}{{V}_{ex} ( T )} - \frac{S}{{V}_{ex} ( T ) + {V}_{in} ( T )} ] & ( 29 )\end{matrix}$$

$$\begin{matrix}[ Formula 38 ] & \\ {I}_{7} = {C}_{e , ex} + \frac{( {C}_{e , in} - {C}_{e , ex} ) Ah}{{V}_{ex} ( T ) \cdot {λ}_{3}} - \frac{S}{{V}_{ex} ( T ) + {V}_{in} ( T )} ( \frac{1}{{λ}_{3}} + T ) & ( 20 )\end{matrix}$$

The above Formulas (3) and (28) to (30) give a function in which time (t) indicating a temporal change in the toxin concentration of the extracellular fluid after dialysis is used as a variable. Determining the time (t) of this function gives the toxin concentration of the extracellular fluid after dialysis at the time (t).

Further, the above Formulas (4) and (28) to (30) give a function in which time (t) indicating a temporal change in the toxin concentration of the intracellular fluid after dialysis is used as a variable. Determining the time (t) of this function gives the toxin concentration of the intracellular fluid after dialysis at the time (t).

### <Corrected Clearance>

For example, the clearance (CL) of Formula (10) can be said to be the performance value of the dialyzer itself. The corrected clearance is a value obtained by adjusting the clearance (CL) using the recirculation rate.

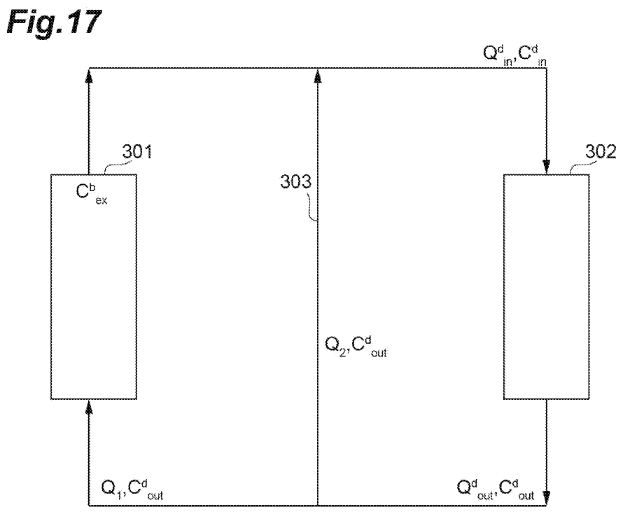

As illustrated in FIG. 17, a circuit including a patient 301 and a dialyzer 302 is set up. Recirculation can be simulated by separating the blood flowing out of the dialyzer 302 into a portion flowing into the patient 301 and a portion flowing into the dialyzer 302 again through a recirculation circuit 303 without passing through the patient 301.

The recirculation rate is defined by Formula (31).

$$\begin{matrix}[ Formula 39 ] & \\ η = \frac{{Q}_{2}}{{Q}_{out}^{d}} & ( 31 )\end{matrix}$$

η: recirculation rate
Q²: Amount of blood to be recirculated
Q^{d}ₒᵤₜ Amount of blood flowing out of the dialyzer 302

The amount of blood (Q^{d}ₒᵤₜ) flowing out of the dialyzer 302 included in Formula (31) is defined by Formula (32).

$$\begin{matrix}[ Formula 40 ] & \\ {Q}_{in}^{d} = {Q}_{out}^{d} + {ω}_{f} & ( 32 )\end{matrix}$$

Q^{d}ᵢₙ: Amount of blood flowing into the dialyzer 302
Q^{d}ₒᵤₜ Amount of blood flowing out of the dialyzer 302
ω_{f}: Amount of liquid removed by the dialyzer 302 (water removal amount).

The clearance (CL) before correction is defined by Formula (33).

$$\begin{matrix}[ Formula 41 ] & \\ CL = \frac{{Q}_{in}^{d} {C}_{in}^{d} - {Q}_{out}^{d} {C}_{out}^{d}}{{C}_{in}^{d}} & ( 33 )\end{matrix}$$

C^{d}ᵢₙ: toxin concentration of blood flowing into the dialyzer 302
C^{d}ₒᵤₜ toxin concentration of blood flowing out of the dialyzer 302

Transforming Formula (33) yields the toxin concentration (C out) of blood flowing out of the dialyzer 302 (Formula 34).

$$\begin{matrix}[ Formula 42 ] & \\ {C}_{out}^{d} = \frac{{Q}_{in}^{d} - CL}{{Q}_{in}^{d} - {ω}_{f}} {Q}_{in}^{d} & ( 34 )\end{matrix}$$

The toxin concentration (C^{d}ᵢₙ) of the blood flowing into the dialyzer 302 of Formula (34) is defined by Formula (35).

$$\begin{matrix}[ Formula 43 ] & \\ {C}_{in}^{d} = \frac{( {Q}_{in}^{d} - {Q}_{2} ) {C}_{ex}^{b} + {θ}_{2} {C}_{out}^{d}}{{Q}_{in}^{d}} & ( 35 )\end{matrix}$$

C^{b}ₑₓ: extracellular fluid toxin concentration

Transforming Formula (35) using Formula (34) yields Formula (36). In Formula (36), the term given to the toxin concentration (Chex) in the blood flowing out of the patient 301 can be defined as a recirculation environment coefficient.

$$\begin{matrix}[ Formula 44 ] & \\ {C}_{in}^{d} = \frac{1 - ( 1 - \frac{{ω}_{f}}{{Q}_{in}^{d}} ) η}{1 - ( 1 - \frac{{ω}_{f}}{{Q}_{in}^{d}} ) ( \frac{{Q}_{in}^{d} - CL}{{Q}_{in}^{d} - {ω}_{f}} ) η} {C}_{ex}^{b} & ( 36 )\end{matrix}$$

From (36), the corrected clearance is defined by Formula (37) including the recirculation rate (η).

$$\begin{matrix}[ Formula 45 ] & \\ Corrected Clearance = \frac{1 - ( 1 - \frac{{ω}_{f}}{{Q}_{in}^{d}} ) η}{1 - ( 1 - \frac{{ω}_{f}}{{Q}_{in}^{d}} ) ( \frac{{Q}_{in}^{d} - CL}{{Q}_{in}^{d} - {ω}_{f}} ) η} CL & ( 37 )\end{matrix}$$

### Reference Example

The usefulness of Formulas (1) to (4) was confirmed. Specifically, the temporal change in the predicted toxin concentration predicted using Formulas (1) to (4) was compared with the temporal change in the test toxin concentration actually obtained by the blood collection.

The test toxin concentrations were obtained from three dialysis patients (hereinafter, referred to as “patient A”, “patient B”, and “patient C”). The test periods for test toxin concentration were during dialysis and 60 minutes after dialysis. The dialysis time was 240 minutes. During the test period, blood was drawn approximately every 20 minutes to obtain toxin concentrations. As the toxin concentration, urea nitrogen was selected.

As the predicted toxin concentration, first, the test toxin concentration was used to obtain a patient-specific value for each patient. Next, the patient-specific value and the dialysis conditional value shown below were used to obtain the predicted toxin concentration for each patient. The coefficient (¿) is the water content ratio between the extracellular fluid and the intracellular fluid.
Dialysis time (T): 240 [min]
Coefficient (ε): 0.667 [-]
Extracellular fluid volume (Vex (0)): 14400 [ml]
Intracellular fluid volume (Vin (0)): 21600 [ml]
Water removal (ω_{f}): 20 [ml/min]
Plasma filling amount (ωₚₗ): 12 [ml/min].

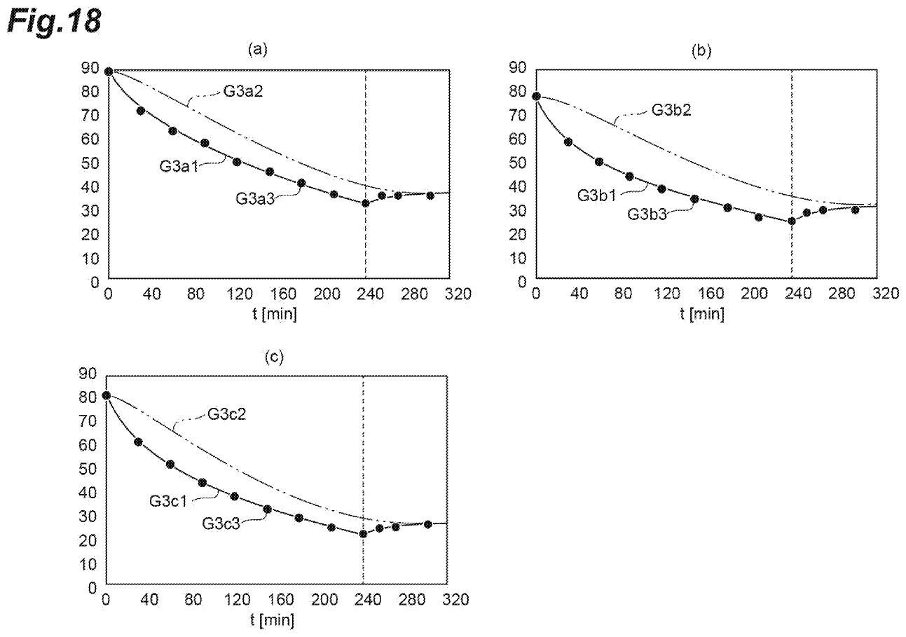

FIG. 18(a) illustrates the test toxin concentration and the predicted toxin concentration of the patient A. FIG. 18(b) illustrates the test toxin concentration and the predicted toxin concentration of the patient B. FIG. 18(c) illustrates the test toxin concentration and the predicted toxin concentration of the patient C. The horizontal axis represents an elapsed time. The vertical axis represents the concentration of urea nitrogen. Graphs G3al, G3b1, and G3cl each show the toxin concentration in the extracellular fluid. Graphs G3a2, G3b2, and G3c2 each show the toxin concentration in the intracellular fluid. Plots G3a3, G3b3, and G3c3 each show the test toxin concentration.

For example, attention is focused on the graph G3al showing the toxin concentration in the extracellular fluid in FIG. 18(a). It was found that the tendency of the temporal change shown in the graph G3al could well predict the temporal change shown in the plurality of plots G3a3 indicating the test toxin concentration. This result indicates that Formulas (1) and (3) for deriving the graph G3al are proper. This result further indicates that the patient-specific values substituted into Formulas (1) and (3) for deriving the graph G3al correctly represent the dialysis-related characteristics of the patient A. In essence, according to Formulas (1) to (4), it was found that the mass transfer characteristics in the living body of the individual patient, which cannot be predicted from the one-compartment model, could be quantified.

### REFERENCE SIGNS LIST

1, 1A DIALYSIS INFORMATION PROVIDING DEVICE
2 INPUT UNIT
3 DISPLAY UNIT
4 PATIENT-SPECIFIC VALUE PROPOSAL UNIT
41 TEST TOXIN CONCENTRATION PROCESSING UNIT
42 PREDICTED TOXIN CONCENTRATION PROCESSING UNIT
43 PATIENT-SPECIFIC VALUE POST-PROCESSING UNIT
421 PATIENT-SPECIFIC VALUE PRE-PROCESSING UNIT
421a PATIENT-SPECIFIC VALUE RECEIVING UNIT
421b PATIENT TEST VALUE RECEIVING UNIT
421c DIALYSIS CONDITIONAL VALUE RECEIVING UNIT
421d COEFFICIENT CALCULATION UNIT
422 TOXIN CONCENTRATION CALCULATION UNIT
422a DURING-DIALYSIS EXTRACELLULAR FLUID CALCULATION UNIT
422b DURING-DIALYSIS INTRACELLULAR FLUID CALCULATION UNIT
422c POST-DIALYSIS EXTRACELLULAR FLUID CALCULATION UNIT
422d POST-DIALYSIS INTRACELLULAR FLUID CALCULATION UNIT
5, 5A SEARCH DIALYSIS CONDITIONAL VALUE PROPOSAL UNIT
51 DIALYSIS CONDITIONAL VALUE PRE-PROCESSING UNIT
52 SEARCH DIALYSIS CONDITIONAL VALUE ACQUISITION UNIT
53 TOXIN CONCENTRATION CALCULATION UNIT
54 CORRECTION NECESSITY DETERMINATION UNIT
55 DIALYSIS CONDITIONAL VALUE REVIEW UNIT
511 PATIENT-SPECIFIC VALUE RECEIVING UNIT
512 PATIENT TEST VALUE RECEIVING UNIT
513 TARGET DIALYSIS CONDITIONAL VALUE RECEIVING UNIT
514 KNOWN DIALYSIS CONDITIONAL VALUE RECEIVING UNIT
521 CONDITION SETTING UNIT
522 DETERMINATION UNIT

1. A device for providing dialysis information, the device providing a search dialysis conditional value that is a value selected from among a plurality of dialysis conditional values indicating conditions for dialysis and satisfies a target for the dialysis, the device comprising:
at least one processor, wherein
the at least one processor
receives a target dialysis conditional value indicating the target for the dialysis among the plurality of dialysis conditions, a plurality of known dialysis conditional values excluding the search dialysis conditional value and the target dialysis conditional value from the plurality of dialysis conditional values, and one or a plurality of patient-specific values set for each patient, and
obtains the search dialysis conditional value satisfying the target dialysis conditional value by using a predicted toxin concentration obtained by substituting the known dialysis condition and the patient-specific value into an extracellular fluid toxin concentration function indicating a temporal change in concentration of a toxin to be removed contained in extracellular fluid of the patient.

2. The device for providing dialysis information according to claim 1, wherein the at least one processor
receives the patient-specific value and the dialysis condition to calculate information indicating a temporal change in concentration of the toxin to be removed by substituting the patient-specific value and the dialysis condition into the extracellular fluid toxin concentration function, and
outputs a concentration of the toxin to be removed contained in the extracellular fluid at a predetermined time point after the dialysis.

3. The device for providing dialysis information according to claim 1, wherein the at least one processor
receives the patient-specific value and the dialysis condition to calculate a concentration of the toxin to be removed by substituting the patient-specific value and the dialysis condition into the extracellular fluid toxin concentration function, and
outputs a concentration of the toxin to be removed contained in the extracellular fluid at a predetermined time point during the dialysis.

4. The device for providing dialysis information according to claim 1, wherein the at least one processor
determines whether the search dialysis conditional value satisfying the target dialysis conditional value has been obtained, and
uses a result of the determination to determine whether the dialysis conditional value to be corrected is present among the plurality of received dialysis conditional values.

5. The device for providing dialysis information according to claim 1, wherein the patient-specific value includes a recirculation rate, and
the at least one processor calculates the concentration of the toxin to be removed by using also the recirculation rate, in addition to the patient-specific value and the dialysis condition, for the extracellular fluid toxin concentration function.

6. The device for providing dialysis information according to claim 1, wherein the search dialysis conditional value is a value of clearance.

7. The device for providing dialysis information according to claim 1, wherein the search dialysis conditional value is a value of an index (Kt/V) defined by clearance, a dialysis time, and a total volume of body fluid.

8. The device for providing dialysis information according to claim 1, wherein the at least one processor outputs information for obtaining the patient-specific value by using the extracellular fluid toxin concentration function.

9. The device for providing dialysis information according to claim 8, wherein the at least one processor
receives a test toxin concentration obtained by testing a concentration of the toxin to be removed contained in the extracellular fluid of the patient in a period in which the dialysis is performed and a concentration of the toxin to be removed contained in the extracellular fluid of the patient in a period after the dialysis is completed, and
predicts a predicted toxin concentration indicating a temporal change in the concentration of the toxin to be removed contained in the extracellular fluid by using the extracellular fluid toxin concentration function.

10. A device for providing dialysis information, the device obtaining one or a plurality of patient-specific values set for each patient undergoing dialysis, the device comprising:
at least one processor, wherein
the at least one processor
receives a test toxin concentration obtained by testing a concentration of a toxin to be removed contained in an extracellular fluid of the patient in a period in which the dialysis is performed and a concentration of the toxin to be removed contained in the extracellular fluid of the patient in a period after the dialysis is completed, and
obtains a predicted toxin concentration indicating a temporal change in the concentration of the toxin to be removed contained in the extracellular fluid by substituting a plurality of dialysis conditional values indicating conditions for the dialysis into an extracellular fluid toxin concentration function indicating a temporal change in the concentration of the toxin to be removed contained in the extracellular fluid of the patient.

11. A program for providing dialysis information, the program providing a search dialysis conditional value that is a value selected from among a plurality of dialysis conditional values indicating conditions for dialysis and satisfies a target for the dialysis, the program causing a computer to function as:
a dialysis conditional value pre-processing unit configured to receive a target dialysis conditional value indicating the target for the dialysis among the plurality of dialysis conditions, a plurality of known dialysis conditional values excluding the search dialysis conditional value and the target dialysis conditional value from the plurality of dialysis conditional values, and one or a plurality of patient-specific values set for each patient; and
a search dialysis conditional value acquisition unit configured to obtain the search dialysis conditional value satisfying the target dialysis conditional value by using a predicted toxin concentration obtained by substituting the known dialysis condition and the patient-specific value into an extracellular fluid toxin concentration function indicating a temporal change in concentration of a toxin to be removed contained in extracellular fluid of the patient.

12. The program for providing dialysis information according to claim 11, the program causing the computer to further function as:
a toxin concentration calculation unit configured to receive the patient-specific value and the dialysis condition transmitted from the dialysis conditional value pre-processing unit to calculate a concentration of the toxin to be removed by substituting the patient-specific value and the dialysis condition into the extracellular fluid toxin concentration function, wherein
the toxin concentration calculation unit outputs a concentration of the toxin to be removed contained in the extracellular fluid at a predetermined time point after the dialysis.

13. The program for providing dialysis information according to claim 11, the program causing the computer to further function as:
a toxin concentration calculation unit configured to receive the patient-specific value and the dialysis condition transmitted from the dialysis conditional value pre-processing unit to calculate a concentration of the toxin to be removed by substituting the patient-specific value and the dialysis condition into the extracellular fluid toxin concentration function, wherein
the toxin concentration calculation unit outputs a concentration of the toxin to be removed contained in the extracellular fluid at a predetermined time point during the dialysis.

14. The program for providing dialysis information according to claim 12, the program causing the computer to further function as:
a determination unit configured to determine whether the search dialysis conditional value satisfying the target dialysis conditional value has been obtained, and
a correction necessity determination unit configured to use a result of the determination to determine whether the dialysis conditional value to be corrected is present among the plurality of received dialysis conditional values.

15. The program for providing dialysis information according to claim 12, wherein the patient-specific value includes a recirculation rate, and
the toxin concentration calculation unit calculates the concentration of the toxin to be removed by using also the recirculation rate, in addition to the patient-specific value and the dialysis condition, for the extracellular fluid toxin concentration function.

16. The program for providing dialysis information according to claim 11, wherein the search dialysis conditional value is a value of clearance.

17. The program for providing dialysis information according to claim 11, wherein the search dialysis conditional value is a value of an index (Kt/V) defined by clearance, a dialysis time, and a total volume of body fluid.

18. The program for providing dialysis information according to claim 11, the program causing the computer to further function as a patient-specific value proposal unit configured to output information for obtaining the patient-specific value by using the extracellular fluid toxin concentration function.

19. The program for providing dialysis information according to claim 18, the program causing the computer to further function as:
a test toxin concentration processing unit configured to receive a test toxin concentration obtained by testing a concentration of the toxin to be removed contained in the extracellular fluid of the patient in a period in which the dialysis is performed and a concentration of the toxin to be removed contained in the extracellular fluid of the patient in a period after the dialysis is completed, and
a predicted toxin concentration processing unit configured to predict a predicted toxin concentration indicating a temporal change in the concentration of the toxin to be removed contained in the extracellular fluid by using the extracellular fluid toxin concentration function.

20. (canceled)
